(19) United States
(12) Patent Application Publication
(10) Pub. No.: US 2013/0209554 A1
(43) Pub. Date: Aug. 15, 2013

(54) NOVEL GELATIN CAPSULE COMPOSITIONS AND METHODS OF MAKING

(71) Applicants: Tom Keenan, Sioux City, IA (US); John M. Dolphin, Sioux City, IA (US)

(72) Inventors: Tom Keenan, Sioux City, IA (US); John M. Dolphin, Sioux City, IA (US)

(73) Assignee: Gelita, Eberbach (DE)

(21) Appl. No.: 13/538,632

(22) Filed: Jun. 29, 2012

Related U.S. Application Data
(63) Continuation-in-part of application No. 13/030,679, filed on Feb. 18, 2011.
(60) Provisional application No. 61/306,744, filed on Feb. 22, 2010.
(60) Provisional application No. 61/503,749, filed on Jul. 1, 2011.

(51) Int. Cl. A61K 9/48

(52) U.S. Cl. CPC A61K 9/4825
USPC 424/456; 424/400; 427/2.21

(57) ABSTRACT

A gelatin capsule composition that includes one or more first distinct regions comprising a gelatin and one or more second distinct regions comprising a water-insoluble rapid-release agent is provided.

### CROSS-REFERENCE

This is a continuation-in-part application of U.S. patent application Ser. No. 13/030,679 filed on Feb. 18, 2011, which claims priority to provisional patent application Ser. No. 61/306,744 filed on Feb. 22, 1010, and the present application claims priority to U.S. provisional application Ser. No. 61/503,749, filed Jul. 1, 2011, the contents of which are hereby incorporated by reference in its entirety.

### FIELD OF THE INVENTION

A gelatin capsule composition including a gelatin component and a rapid-release agent is provided. In particular the current invention is directed to a novel gelatin capsule composition that includes a rapid-release agent spread throughout a gelatin component coating.

### BACKGROUND OF THE INVENTION

The development of compositions for the rapid release of active pharmaceutical compounds (APIs) is an ongoing challenge in the pharmaceutical industry. Due to various unpalatable characteristics of many APIs such as bitterness, some oral compositions incorporate added flavorants to mask the unpalatable flavor of the API. Further, other oral pharmaceutical compositions include an encapsulant to isolate the API during oral administration, to alleviate other unpalatable characteristics of the API such as grittiness or stickiness, and to enhance other properties of the composition such as stability during storage and/or transport. Encapsulated pharmaceutical compositions such as gelatin-coated tablets, hard capsules, and soft gelatin capsules are widely used for orally administered therapeutic compositions.

Although the encapsulation of APIs alleviates many of the palatability and stability issues described above, the encapsulation material properties pose a further challenge with respect to the rapid release of the API into the gastric cavity. Some existing approaches make use of a water-soluble encapsulant material, but many water-soluble encapsulants tend to partially dissolve in the oral cavity or esophagus causing sticking during oral administration. Other approaches make use of pH-sensitive coatings that incorporate polymeric materials that are relatively insoluble in a relatively neutral pH environment such as the oral cavity, but are highly soluble in an acidic environment such as the gastric cavity; however, the production of pH-sensitive polymer encapsulants typically requires specialized production techniques.

Other existing rapid-release compositions incorporate various water-soluble porigenic materials into the encapsulation material that dissolve in the gastric cavity and form pores in the remaining encapsulation material, allowing the API within to dissolve and diffuse outward into the gastric cavity. Yet other existing rapid-release compositions include a multilayer encapsulant in which a waterproof porous outer layer conducts gastric juices to a swellable inner layer material, which bursts the encapsulant upon swelling and exposes the underlying API. These encapsulant compositions require considerable effort to manufacture and require the penetration of the gastric juices through pores in an outer coating layer to implement the release of the API, resulting in delayed release times for the APIs in the gastric cavity.

Gelatin is a well-established encapsulation material in the pharmaceutical industry. The material properties of gelatin may be adjusted or controlled by gelatin treatment methods such as cross-linking, deionization and partial hydrolysis to produce gelatin coatings with specified material properties such as rigidity and solubility. The production of gelatin coatings, soft gelatin capsules, and hard gelatin capsules typically utilizes an aqueous suspension of the gelatin. As a result, it is difficult to incorporate water-soluble rapid-release additives to enhance the rapid-release properties of the resulting gelatin encapsulant in the gastric cavity. Further, gelatin rapid-release additives may alter the chemical properties of the gelatin suspension such as pH, which may cause degradation or instability of the resulting gelatin matrix structure.

A need exists in the art for a gelatin-based gelatin capsule composition that rapidly degrades in the acidic environment of the gastric cavity, but not in pH-neutral aqueous environment of the oral cavity and esophagus during oral administration of the API. Further, a need in the art exists for rapid-release additives to enhance the rapid-release properties of gelatin or other polymer encapsulants in the gastric cavity that do not degrade the encapsulant's matrix structure during production. Such a rapid-release additive would facilitate the production of gelatin capsules using well-established encapsulation technologies.

### SUMMARY OF INVENTION

The presently claimed embodiments of the current invention generally provide a gelatin capsule formulation having distinct regions with variable dissolution and release profiles, providing rapid dissolution and release of the contents of the composition. Accordingly, among the many aspects of the current invention is a gelatin capsule composition comprising an outer surface coating and an inner core, the outer surface coating comprising: (a) one or more first distinct regions comprising a gelatin component; and, (b) one or more second distinct regions comprising a rapid-release agent. In one embodiment, the one or more first distinct regions comprising a gelatin component and the one or more second distinct regions comprising a rapid-release agent further comprise a gelatin hydrolysate, having an average molecular weight ranging from about 100 to about 2000 Daltons. The gelatin component may have an average molecular weight ranging from about 50,000 Daltons to about 300,000 Daltons.

In another embodiment, the one or more second distinct regions comprising a rapid-release agent span the depth of the outer surface coating from the proximal surface to the distal surface, and the one or more second distinct regions comprising a rapid-release agent may be dispersed across the gelatin capsule composition in a uniform or non-uniform pattern.

In another embodiment, the rapid-release agent may include a water-insoluble carbonate salt, a water-soluble carbonate salt, and combinations thereof. The water-insoluble carbonate salt may include bismuth subcarbonate, calcium carbonate, cobalt carbonate, lanthanum carbonate, lead carbonate, lithium carbonate, magnesium carbonate, manganese carbonate, nickel (II) carbonate, silver carbonate, strontium carbonate, and combinations thereof. In a further embodiment, the water-insoluble carbonate salt may be essentially insoluble at a pH ranging from about 6 to about 8, and may dissociate at a pH ranging from 0 to about 3. The water-soluble carbonate salt may include sodium carbonate, potassium carbonate, ammonium carbonate, sodium bicarbonate, potassium bicarbonate, ammonium bicarbonate, lithium carbonate, and combinations thereof, and may be at least partially soluble at a pH ranging from about 0 to about 3.

In another embodiment, the gelatin composition may include a mass ratio of the rapid-release agent to the gelatin component ranging from about 1:1 to about 1:20 and about 1:2 to about 1:9. In certain embodiments, the gelatin capsule composition degrades essentially completely in less than 15 minutes at a pH ranging between 0 and about 3.

Further, in another embodiment, the outer surface coating may also include a plasticizer including dibutyl sebacate, diethyl phthalate, glycerine, polyethylene glycol, propylene glycol, sorbitol, erythritol, triacetin, triethyl citrate, water, and mixtures thereof. In one embodiment, the plasticizer may include a combination of sorbitol, sorbitan, glycerine, and water. The gelatin component incorporated into the outer surface coating may include a combination of a gelatin, a plasticizer, and water. Specifically, in one embodiment, the one or more first distinct regions comprise about 25% to about 55% by weight of the one or more first distinct regions of the gelatin, about 10% to about 30% by weight of the one or more first distinct regions of the plasticizer, and about 15% to about 45% by weight of the one or more first distinct regions of water. In another embodiment, the gelatin component of the one or more first distinct regions comprise about 35% to about 45% by weight of the one or more first distinct regions of the gelatin, about 16% to about 24% by weight of the one or more first distinct regions of the plasticizer, and about 20% to about 30% by weight of the one or more first distinct regions of the water.

In an alternative embodiment, the gelatin component may incorporate a combination of a gelatin and water, including from about 5% to about 30% gelatin and from about 70% to about 95% water. The gelatin component may also include about 10% to about 20% gelatin and about 80% to about 90% water. Moreover, in a further embodiment, the outer surface coating may include a mass ratio of the gelatin component to the gelatin hydrolysate ranging from about 3:1 to about 99:1, and from about 4:1 to about 19:1.

In an additional embodiment, the one or more second distinct regions comprising a rapid-release agent may further include a gelatin, a plasticizer, and water. In one embodiment, the one or more second distinct regions comprise about 20% to about 50% by weight of the one or more second distinct regions of the gelatin, about 1% to about 25% by weight of the one or more second distinct regions of the rapid-release agent, about 10% to about 30% by weight of the one or more second distinct regions of the plasticizer, and about 20% to about 40% by weight of the one or more second distinct regions of water. In another embodiment, the one or more second distinct regions comprise about 30% to about 45% by weight of the one or more second distinct regions of the gelatin, about 4% to about 16% by weight of the one or more second distinct regions of the rapid-release agent, about 16% to about 24% by weight of the one or more second distinct regions of the plasticizer, and about 26% to about 34% by weight of the one or more second distinct regions of water.

The inner core component of the currently claimed embodiment may include either a solid formulation such as a tablet, capsule, beads, and granules, a paste, or a liquid formulation. Further, the inner core may include an active pharmaceutical ingredient, and, optionally, one or more pharmaceutically acceptable excipients.

In another embodiment, the one or more first distinct regions may also include a rapid release agent, wherein the rapid release agent is present in a concentration less than the concentration of the rapid release agent of the one or more second distinct regions. Moreover, the gelatin component may generally include Type A gelatin, Type B gelatin, and combinations thereof.

In additional embodiments, the one or more second distinct regions including the rapid-release agent substantially dissolve in about 30 minutes or less, in about 20 minutes or less, in about 15 minutes or less, in about 12 minutes or less, in about 10 minutes or less, in about 8 minutes or less, in about 6 minutes or less, and in about 5 minutes or less, thereby releasing the contents of the inner core. As used herein, the term “substantially dissolve” and “degrade essentially completely” are interchangeable, and are defined to require that at least a portion of the one or more second distinct regions has dissolved from the distal surface of the outer surface coating to the proximal surface of the outer surface coating, resulting in at least a portion of the core being exposed to the external environment, allowing the release of the core contents. Further, the one or more second distinct regions comprising a rapid-release agent may have the appearance of stripes, bars, bands, streaks, strips, rows, columns, spots, flecks, striations, belts, ribbons, veins, dashes, ridges, strains, and combinations thereof. The one or more second distinct regions may comprise uniform and non-uniform shapes. In one embodiment the one or more second distinct regions comprise a uniform shape, whereby the stripes, bars, bands, etc. have a consistent look and the length (as measured in relation to the circumference along one axis of the composition) and the width (as measured in relation to the circumference along an alternative axis perpendicular to the axis used to define the length) do not vary more than 10% relative to each distinct region. In another embodiment, the one or more distinct regions comprise a non-uniform shape, whereby the stripes, bars, bands, etc. have a distinct look and shape, and the length (as measured in relation to the circumference along one axis of the composition) and the width (as measured in relation to the circumference along an alternative axis perpendicular to the axis used to define the length) vary more than 10% relative to each distinct region.

In another aspect, the currently claimed embodiments include a gelatin capsule composition comprising an outer surface coating and an inner core, the outer surface coating comprising: (a) one or more first distinct regions comprising a gelatin component and a gelatin hydrolysate; and, (b) one or more second distinct regions comprising a rapid-release agent, a gelatin component, and a gelatin hydrolysate, wherein the one or more second distinct regions span the depth of the outer surface coating from the proximal surface to the distal surface.

In yet another aspect, the currently claimed embodiments include a pharmaceutical gelatin capsule composition comprising an outer surface coating and an inner core, the outer surface coating comprising: (a) one or more first distinct regions comprising about 35% to about 45% by weight of the one or more first distinct regions of a gelatin, about 3% to about 7% by weight of the one or more first distinct regions of a gelatin hydrolysate having an average molecular weight ranging from about 100 to about 2000 Daltons, about 18% to about 22% by weight of the one or more first distinct regions of a plasticizer, and about 20% to about 26% by weight of the one or more first distinct regions of water; and, (b) one or more second distinct regions comprising about 34% to about 40% by weight of the one or more second distinct regions of a gelatin, about 3% to about 7% by weight of the one or more second distinct regions of a gelatin hydrolysate having an average molecular weight ranging from about 100 to about 2000 Daltons, about 5% to about 10% by weight of the one or more second distinct regions of a rapid release agent, about 18% to about 22% by weight of the one or more second distinct regions of a plasticizer, and about 28% to about 32% by weight of the one or more second distinct regions of water, wherein the inner core comprises an active pharmaceutical ingredient, wherein the one or more second distinct regions span the depth of the outer surface coating from the proximal surface to the distal surface, and, wherein the rapid release agent comprises bismuth subcarbonate, calcium carbonate, cobalt carbonate, lanthanum carbonate, lead carbonate, lithium carbonate, magnesium carbonate, manganese carbonate, nickel (II) carbonate, silver carbonate, strontium carbonate, sodium carbonate, potassium carbonate, ammonium carbonate, sodium bicarbonate, potassium bicarbonate, ammonium bicarbonate, and combinations thereof.

In still another aspect, the currently claimed embodiments of the invention include a gelatin capsule composition comprising an outer surface coating, a subcoating, and an inner core, the outer surface coating comprising: (a) one or more first distinct regions comprising a gelatin component; and, (b) one or more second distinct regions comprising a rapid-release agent, wherein the subcoating is applied to the distal portion of the core and surrounds a substantial portion of the core, and wherein the outer surface coating is applied to the distal portion of the subcoating. In one embodiment, the subcoating may generally include a gelatin component, a gelatin hydrolysate, a polymeric material, and combinations thereof. Further, in another embodiment, the subcoating may also include one or more passageways for the release of the contents of the inner core. In a further embodiment, the subcoating may include two or more layers having distinct release and dissolution profiles. In one embodiment, the inner core comprises an active pharmaceutical ingredient.

In a further aspect, the currently claimed embodiments encompass a gelatin capsule composition comprising an enteric coating, an inner core surface coating, and an inner core, the inner core surface coating comprising: (a) one or more first distinct regions comprising a gelatin component; and, (b) one or more second distinct regions comprising a rapid-release agent, wherein the enteric coating is applied to the distal portion of the inner core surface coating, wherein the inner core surface coating is applied to the distal portion of the inner core, and wherein the enteric coating is not soluble at pH levels ranging from 0 to about 3 and dissolves at pH levels of greater than or equal to 5.5. In one embodiment, the enteric coating may include a gelatin component, a gelatin hydrolysate, a polymeric material, and combinations thereof. Also, the inner core may incorporate an active pharmaceutical ingredient.

In still another aspect, the currently claimed embodiments include a method for manufacturing a gelatin capsule composition comprising an outer surface coating and an inner core, wherein the outer surface coating includes one or more first distinct regions comprising a gelatin component, and one or more second distinct regions comprising a rapid-release agent, the method comprising: (a) dissolving a gelatin component in an aqueous medium to create an aqueous gelatin solution; (b) incorporating a second solution comprising a rapid-release agent into the aqueous gelatin solution to produce one or more first distinct regions comprising the aqueous gelatin solution and one or more second distinct regions comprising the second solution comprising the rapid-release agent; and, (c) encapsulating an active pharmaceutical ingredient while maintaining the one or more first distinct regions and the one or more second distinct regions. In one embodiment, the encapsulation process of step (c) comprises an extrusion process. In another embodiment, the gelatin component of step (a) comprises a combination of a gelatin having a molecular weight ranging from about 50,000 Daltons to about 300,000 Daltons and a gelatin hydrolysate having a molecular weight ranging from about 100 Daltons to about 2000 Daltons.

Further, step (a) may also include the addition of a plasticizer such as dibutyl sebacate, diethyl phthalate, glycerine, polyethylene glycol, propylene glycol, sorbitol, erythritol, triacetin, and triethyl citrate, water, and mixtures thereof. In one embodiment, the plasticizer may include a combination of sorbitol, glycerine, and water. In another embodiment, the plasticizer component including the combination of sorbitol, sorbitans, glycerine, and water incorporates 50 parts of a combination of sorbitol and sorbitans, 42.5 parts glycerine, and 7.5 parts water. In another embodiment, the gelatin component may include a combination of a gelatin and a plasticizer.

In another embodiment, step (a) may include mixing about 25% to about 55% by weight of the gelatin component of a gelatin, about 15% to about 30% by weight of the gelatin component of a plasticizer, and about 25% to about 40% by weight of the gelatin component of water.

In an additional embodiment, the second solution of step (b) may include a rapid-release agent, a gelatin component, a plasticizer, and an aqueous medium, such as water. The gelatin component may incorporate a combination of a gelatin having a molecular weight ranging from about 50,000 Daltons to about 300,000 Daltons and a gelatin hydrolysate having a molecular weight ranging from about 100 Daltons to about 2000 Daltons.

In yet another embodiment, the one or more first distinct regions comprising an aqueous gelatin solution and the one or more second distinct regions comprising a rapid-release agent are produced by feeding the aqueous gelatin solution and the second solution comprising the rapid-release agent through a static mixer.

In still another aspect, the currently claimed embodiments include a gelatin composition comprising an outer surface coating and an inner core, wherein the outer surface coating comprises a rapid-release agent and a gelatin component, wherein rapid-release agent is at least semi-homogenously dispersed throughout the outer surface coating, without distinct regions. The gelatin composition may further include a gelatin hydrolysate having a molecular weight ranging from about 100 Daltons to about 2000 Daltons.

Moreover, in one embodiment, the rapid-release agent comprises water-soluble carbonate salts, water-insoluble carbonate salts, and combinations thereof. The water-soluble rapid release agent may generally include sodium carbonate, potassium carbonate, ammonium carbonate, sodium bicarbonate, potassium bicarbonate, ammonium bicarbonate, and combinations thereof, and may be at least partially soluble in water, and wherein the rapid-release agent dissociates at a pH ranging from 0 to about 3. In addition, the water-insoluble carbonate salt may generally include bismuth subcarbonate, calcium carbonate, cobalt carbonate, lanthanum carbonate, lead carbonate, lithium carbonate, magnesium carbonate, manganese carbonate, nickel (II) carbonate, silver carbonate, strontium carbonate, and combinations thereof.

In certain embodiments, the composition may include a mass ratio of the rapid-release agent to the gelatin component ranging from about 1:1 to about 1:20, from about 1:2 to about 1:15, and from about 1:4 to about 1:9. Further, in another embodiment, the composition degrades essentially completely in less than 15 minutes at a pH ranging between 0 and about 3.

In a further embodiment, the composition may include a mass ratio of the gelatin component to the gelatin hydrolysate ranging from about 3:1 to about 99:1, from about 4:1 to about 19:1, and from about 5:1 to about 13:1.

In a further aspect, the currently claimed embodiments include a gelatin composition comprising: (a) a water-soluble rapid-release agent; (b) a gelatin component; and (c) a gelatin hydrolysate, wherein the composition includes a mass ratio of the water-soluble rapid-release agent to the gelatin component ranging from about 1:1 to about 1:20, wherein the gelatin hydrolysate has a molecular weight ranging from about 100 Daltons to about 2000 Daltons, and wherein the composition includes a mass ratio of the gelatin component to the gelatin hydrolysate ranging from about 3:1 to about 99:1.

In still another aspect, the currently claimed embodiments include a gelatin composition comprising a rapid-release agent and a gelatin component, prepared by the process comprising: (a) dissolving a gelatin component in an aqueous medium to form an aqueous gelatin solution; (b) mixing a rapid-release agent with the aqueous gelatin solution prior to capsule formation; and (c) incorporating the combination of the aqueous gelatin solution and water-soluble rapid-release agent into a gelatin capsule forming machine. In addition, step (b) may include mixing the rapid-release agent with the aqueous gelatin solution prior to capsule formation comprises an in-line mixing process. In one embodiment, the rapid release composition may further include a gelatin hydrolysate having a molecular weight ranging from about 100 Daltons to about 2000 Daltons.

In yet another aspect, the currently claimed embodiments include a method for manufacturing a gelatin composition comprising a rapid release agent, comprising the steps of: (a) dissolving a gelatin component in an aqueous medium; (b) mixing a rapid-release agent with the aqueous gelatin solution prior to capsule formation; and (c) incorporating the combination of the aqueous gelatin solution and rapid-release agent into a gelatin capsule forming machine. In one embodiment, step (b) may include mixing the rapid-release agent with the aqueous gelatin solution prior to capsule formation using an in-line mixing process. In another embodiment, the gelatin component of step (a) comprises a combination of a gelatin having a molecular weight ranging from about 50,000 Daltons to about 300,000 Daltons and a gelatin hydrolysate having a molecular weight ranging from about 100 Daltons to about 2000 Daltons.

In one specific embodiment, step (a) of the method of making the composition comprises dissolving about 0.01% to about 30% of the gelatin component by weight of the combined aqueous gelatin solution in about 40% to about 99.9% of the aqueous medium by weight of the combined aqueous gelatin solution. In another embodiment, step (a) comprises dissolving about 10% to about 20% of the gelatin component by weight of the combined aqueous gelatin solution in about 70% to about 90% of the aqueous medium by weight of the combined aqueous gelatin solution. In a further embodiment, step (a) comprises dissolving about 10% to about 20% by weight of the gelatin having a molecular weight ranging from about 50,000 Daltons to about 300,000 Daltons and about 1% to about 5% by weight of the gelatin hydrolysate having a molecular weight ranging from about 100 Daltons to about 2000 Daltons in about 70% to about 90% aqueous medium by weight of the combined aqueous gelatin solution.

Other aspects and iterations of the invention are described in detail below.

### DESCRIPTION OF FIGURES

The following figures illustrate various aspects of the invention:

FIG. 1 is a picture of a gelatin composition in accord with the currently claimed embodiments having one distinct region comprising the rapid-release agent and two distinct regions comprising the gelatin component without any rapid-release agent present, wherein the one distinct region comprising a rapid-release agent is of uniform size and shape. The two formulations provide an example of the varying widths of regions that may be incorporated into the claimed compositions.

FIG. 2 is a picture of a gelatin composition in accord with the currently claimed embodiments, wherein one half of the gelatin composition is comprised entirely of a gelatin component absent any substantial amount of rapid-release agent, and the other half comprises one distinct region comprising the rapid-release agent and two distinct regions comprising the gelatin component without any rapid-release agent present.

FIG. 3 is a graph showing the measured dissolution in a pH=1 buffer solution of gelatin compositions that included various acid compounds.

FIG. 4 is a graph showing the effect of adding sodium carbonate on the measured dissolution of a gelatin composition in a pH=1 buffer solution. Specifically, FIG. 4 illustrates the dissolution profile for a composition comprising gelatin and sodium bicarbonate, as compared to a composition comprising only gelatin.

FIG. 5 is a graph showing the effect of storing a gelatin composition on its measured dissolution characteristics in a pH=1 buffer solution.

FIG. 6 is a graph showing the effect of storing a gelatin composition that includes calcium carbonate on its measured dissolution characteristics in a pH=1 buffer solution.

FIG. 7 is a graph showing the effect of storing a gelatin composition that includes calcium carbonate and gelatin hydrolysates on its measured dissolution characteristics in a pH=1 buffer solution.

FIG. 8 is a graph comparing the measured dissolution of three different gelatin compositions in a pH=1 buffer solution after 11 weeks of storage.

FIG. 9 is a graph showing the effect of storing a gelatin composition that includes calcium carbonate on its measured dissolution characteristics in deionized water.

FIG. 10 is a graph showing the effect of storing a gelatin composition that includes calcium carbonate on its measured dissolution characteristics in deionized water.

FIG. 11 is a graph showing the effect of storing a gelatin composition that includes calcium carbonate and gelatin hydrolysates on its measured dissolution characteristics in deionized water.

FIG. 12 is a graph comparing the measured dissolution of three different gelatin compositions in deionized water after 11 weeks of storage.

FIG. 13 illustrates the dissolution profile of the three gelatin formulations in simulated gastric fluid (at a pH of approximately 1.3, and in the absence of any enzymes). The three gelatin formulations include a bone gelatin without any further modifications (“Std Bone Gelatin”); a bone gelatin as well as 15% calcium carbonate (CaCO₃) by weight (“RR only”); and a bone gelatin, 15% calcium carbonate (CaCO₃) by weight, and 10% by weight of hydrolyzed bone gelatin having a molecular weight of about 500 Daltons (“RR RXL”).

FIG. 14 illustrates the dissolution profile of three gelatin formulations in water (approximately neutral pH levels) after the formulations were stored at 40° C. and 75% relative humidity for a period of two weeks. The three gelatin formulations include a bone gelatin without any further modifications (“Std Bone Gelatin”); a bone gelatin as well as 15% calcium carbonate (CaCO₃) by weight (“RR only”); and a bone gelatin, 15% calcium carbonate (CaCO₃) by weight, and 10% by weight of hydrolyzed bone gelatin having a molecular weight of about 500 Daltons (“RR RXL”).

FIG. 15 illustrates the dissolution profile of three gelatin formulations in simulated gastric fluid (at a pH of approximately 1.3, and in the absence of any enzymes), after the formulations were stored at 40° C. and 75% relative humidity for a period of two weeks. The three gelatin formulations include a bone gelatin without any further modifications (“Std Bone Gelatin”); a bone gelatin as well as 15% calcium carbonate (CaCO₃) by weight (“RR only”); and a bone gelatin, 15% calcium carbonate (CaCO₃) by weight, and 10% by weight of hydrolyzed bone gelatin having a molecular weight of about 500 Daltons (“RR RXL”).

FIG. 16 illustrates the dissolution profile of three gelatin formulations in simulated gastric fluid (at a pH of approximately 1.3, and in the absence of any enzymes), after the formulations were stored at 40° C. and 75% relative humidity for a period of four weeks. The three gelatin formulations include a bone gelatin without any further modifications (“Std Bone Gelatin”); a bone gelatin as well as 15% calcium carbonate (CaCO₃) by weight (“RR only”); and a bone gelatin, 15% calcium carbonate (CaCO₃) by weight, and 10% by weight of hydrolyzed bone gelatin having a molecular weight of about 500 Daltons (“RR RXL”)

FIG. 17 is a graph illustrating the dissolution time for four different gelatin compositions including: a gelatin encapsulation composition containing calcium carbonate and sodium carbonate in a mass ratio of approximately 1:1 (6.4% to 6.2%, respectively); a gelatin encapsulation composition containing calcium carbonate and sodium carbonate in a mass ratio of approximately 2.4:1 (11.1% to 4.6%, respectively); a gelatin encapsulation composition containing only sodium carbonate (5.0% concentration); and a gelatin encapsulation composition containing calcium carbonate and sodium carbonate in a mass ratio of approximately 1.5:1 (7.1% to 4.8%, respectively).

FIG. 18 is a graph illustrating the dissolution profile of five gelatin compositions including one composition comprising standard bone gelatin without any rapid release agent (Std Bone Gelatine), one composition comprising standard bone gelatin and calcium carbonate as the rapid release agent (“CaCO3”), one composition comprising standard bone gelatin with an elevated pH from the addition of sodium hydroxide (“HipH”), one composition comprising standard bone gelatin having an elevated pH due to the addition of sodium hydroxide and a narrow width band of standard bone gelatin mixed with calcium carbonate and potassium bicarbonate (“narrow band”), and one composition comprising standard bone gelatin having an elevated pH due to the addition of sodium hydroxide and a medium width band of standard bone gelatin mixed with calcium carbonate and potassium bicarbonate (“medium band”).

### DETAILED DESCRIPTION

As used herein, the term “composition” applies to any solid object, semi-solid, or liquid composition designed to contain a specific pre-determined amount (dose) of a certain ingredient, for example an active ingredient as defined below. Suitable compositions may be pharmaceutical drug delivery systems, including those for oral administration, buccal administration, rectal administration, topical or mucosal delivery, or subcutaneous implants, or other implanted drug delivery systems; or compositions for delivering minerals, vitamins and other nutraceuticals, oral care agents, flavorants, and the like. In one embodiment of the invention, the compositions are considered to be solid; however they may contain liquid or semi-solid components. Generally, the dosage form is an orally administered system for delivering a pharmaceutical active ingredient to the gastro-intestinal tract of a human.

The gelatin composition typically includes an outer surface coating and a core. The core (or substrate) may be any solid, semi-solid, or liquid form. The core may prepared by any suitable method, for example the core be a compressed dosage form, may be molded, or may be injected to provide a liquid filled core. As used herein, “core” refers to a material that is at least partially enveloped or surrounded by another material. In one embodiment, the core comprises a liquid fill formulation generally comprising a solution, suspension, or emulsion. In another embodiment, the core comprises a solid, for example, the core may be a compressed or molded tablet, hard or soft capsule, suppository, or a confectionery form such as a lozenge, nougat, caramel, fondant, or fat based composition.

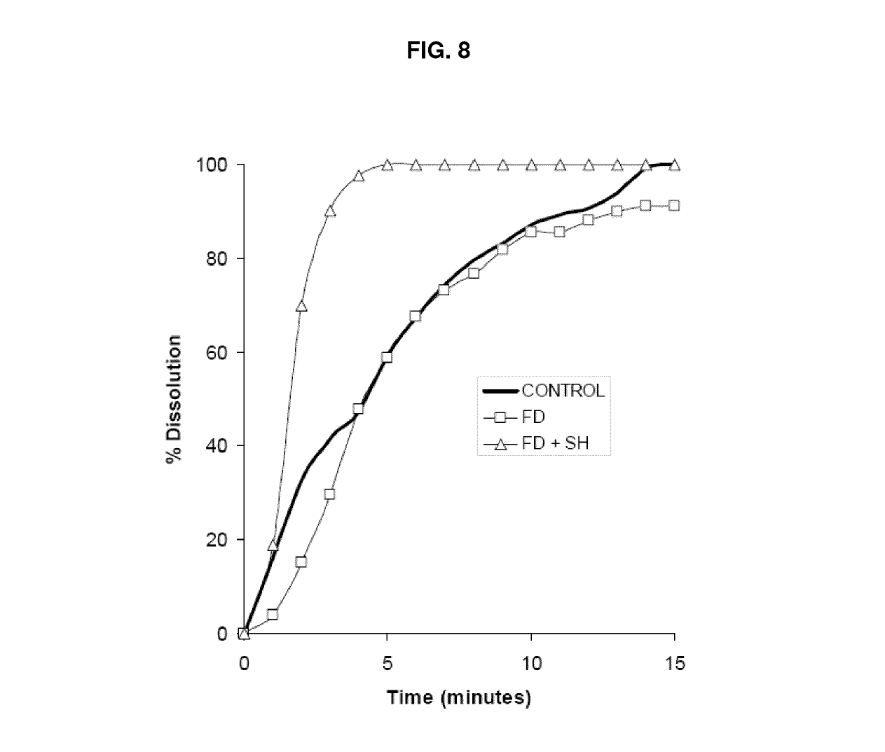

The core may be in a variety of different shapes. For example, in one embodiment the core may be in the shape of a truncated cone. In other embodiments the core may be shaped as a polyhedron, such as a cube, pyramid, prism, or the like; or may have the geometry of a space figure with some non-flat faces, such as a cone, cylinder, sphere, oval, ellipse, torus, or the like. In one embodiment where the core comprises a liquid formulation, the core typically takes the shape of a sphere or oval. In another embodiment, where the core is a solid compressed tablet formulation, the core shapes employed include tablet shapes formed from compression tooling shapes described by “The Elizabeth Companies Tablet Design Training Manual” (Elizabeth Carbide Die Co., Inc., p. 7 (McKeesport, Pa.) (incorporated herein by reference) as follows (the tablet shape corresponds inversely to the shape of the compression tooling): Shallow Concave. Standard Concave. Deep Concave. Extra Deep Concave. Modified Ball Concave. Standard Concave Bisect. Standard Concave Double Bisect. Standard Concave European Bisect. Standard Concave Partial Bisect. Double Radius. Bevel & Concave. Flat Plain. Flat-Faced-Beveled Edge (F.F.B.E.). F.F.B.E. Bisect. F.F.B.E. Double Bisect. Ring. Dimple. Ellipse. Oval. Capsule. Rectangle. Square. Triangle. Hexagon. Pentagon. Octagon. Diamond. Arrowhead. Bullet. Barrel. Half Moon. Shield. Heart. Almond. House/Home Plate. Parallelogram. Trapezoid. FIG. 8/Bar Bell. Bow Tie. Uneven Triangle.

The core may include a blend of suitable active ingredients excipients which may be either their natural color, including white, or can be conventionally colored as desired to provide a core of any desired color.

In one embodiment, the core of the currently claimed gelatin composition may contain one or more active ingredients. As used herein, the term “active ingredient” broadly includes, for example, pharmaceuticals, minerals, vitamins and other nutraceuticals, plant extracts, fatty acids, oral care agents, flavorants and mixtures thereof. Suitable examples active pharmaceutical ingredients include, but are not limited to, abortifacients, ACE inhibitors, adrenocorticotropic hormones, α-adrenergic agonists, α-adrenergic blockers, α-glucosidase inhibitors, anabolic steroids, narcotic analgesics, non-narcotic analgesics, anorexics, antacids, antihelmintics, antiallergics, antialopecials, antiamoebics, antianginals, antiarrhythmics, antiarthritics, antiasthmatics, antibiotics, anticholinergics, anticonvulsants, antidepressants, antidiabetics, antidiarrheals, antidotes, antidyskinetics, antiemetics, antiestrogens, antifungals, antiglaucoma agents, antigout agents, antihistaminics, antihypertensives, nonsteroidal antiinflamatories, antimalarials, antimigraines, antimuscarinics, antinauseants, antineoplastics, antiparkinsonians, antipheochromocytoma agents, antipneumocystis, antiprostatic hyperplasia agent, antiprotozoals, antipruritics, antipsoriatics, antipsychotics, antipyretics, antirickettsials, antispasmodics, antithrombocythemics, antithrombotics, antithyroid agents, antituberculosis agents, antitussives, antiulceratives, antivirals, anxiolytics, aromatase inhibitors, autonomic drugs, barbiturates, benzodiazepine antagonists, β-adrenergic antagonists, β-adrenergic blockers, bradycardic agents, bronchodilators, calcium channel blockers, carbonic anhydrase inhibitors, cardiac drugs, cardiotonics, choleretics, cholinergics, cholinesterase inhibitors, cholinesterase reactivators, CNS stimulants, cytoprotectants, decongestants, diuretics, dopamine receptor agonists, dopamine receptor antagonists, ectoparasiticides, emetics, expectorants, fibrinogen receptor antagonists, gastric secretion inhibitors, gastrointestinal drugs, gastroprokinetics, genitourinary smooth muscle relaxants, heavy metal antagonists, hemostatics, histamine H2 receptor antagonists, hypnotics, immunomodulators, immunosuppressants, iron preparations, keratolytics, MAO inhibitors, mucolytics, muscle relaxants, mydriatics, narcotic antagonists, nootropics, opiate agonists, oxytocics, potassium channel activators, respiratory stimulants, sedatives, serenics, serotonin receptor agonists, serotonin receptor antagonists, serotonin uptake inhibitors, stimulants, sympatholytic agents, sympathomimetics, thrombolytics, tocolytics, tranquilizers, vasodilators, vasoprotectants, and vitamins.

In one embodiment, the active pharmaceutical ingredient (API) may be water-soluble in aqueous solutions having a pH ranging from 0 to about 9. Alternatively, the water-soluble APIs may be water-soluble in aqueous solutions having a pH ranging from 0 to about 3, including, but not limited to, gastric juices. Non-limiting examples of water soluble APIs include abacavir sulfate, acebutolol, acetaminophen, acyclovir, albendazole, alendronate sodium, allopurinol, amoxicillin, amantadine HCl, aminobenzoate potassium, aminocaproic acid, aminoarone HCl, amitriptyline hydrochloride, amphetamine, aspirin, atenolol, atorvastatin calcium, atropine sulfate, azithromycin, balsalazide, benzepril hydrochloride, bepridil HCl, betaine HCl, bisoprolol fumarate, buformin, bupropion HCl, calacyclovir, capecitabine, captopril, carisoprodol, cefadroxil, cefdnir, cefixime, cefpodoxime proxetil, cefprozil, cefuroxime axetil, celecoxib, cetrizine hydrochloride, chondroitin, chlorathiazide, chlorpheniramine maleate, chlorpromazine HCl, chlorzoxazone, choline magnesium trisalicylate, cimetidine, ciprofloxacin, clavulanate potassium, clindamycin, clomipramine hydrochloride, clonidine hydrochloride, clopidogrel bisulfate, cloxacillin sodium, codeine phosphate, colchicines, colsevelam HCl, creatine, cyclophosphamide, cyproheptadine, delavirdine mesylate, demeclocycline HCl, diclofenac, didanosine, diethylcarbamazine citrate, diltiazem HCl, DL-methionine, doxepine HCl, doxycycline, efavirenz, eprosartan mesylate, entacapone, ethembutol hydrochloride, eprosartan, erythromycin, ethosuximide, etidronate disodium, etodolac, ferrous sulfate, flecamide acetate, felbamate, fexofenadine HCl, firocoxib, fluconazole, fluoxetine hydrochloride, fluriprofen, fluvastatin, fosonopril sodium, fumarate, gabapentine, gatifloxacin, ganciclovir, guaifenesine, hydralazine hydrochloride, hydrocodone bitartrate, hydroxychloroquine sulfate, hydroxyurea, hydroxyzine hydrochloride, ibuprofen, indinavir sulfate, irbesartan, isoflavone, isoniazid, isosorbide mononitrate, ketoprofen, lactobionate, lamivudine, levamisole hydrochloride, levofloxacin, lisinopril, lithium carbonate, losartan potassium, mebendazole, mefenamic acid, meperidine HCl, mesalamine, metaprolol tartrate, metaxalone, metformin HCl, methenamine mandelate, methyldopa, methocarbamol, methylphenidate, methylphenidate hydrochloride, metyrosine, minocycline hydrochloride, modafinil, montelukast sodium, morphine sulfate, moxifloxacin HCl, mycophenolate mefetil, nabumetone, naproxen sodium, nefazodone HCl, nelfinavir meslyate, neostigmine bromide, niacin, nicotinamide, nitrofurantoin, nifurtimox, nizatidine, norfloxacin, nortriptyline hydrochloride, ofloxacin, olanzepine, orlistat, oxybytynin chloride, pancreatin, pantothenic acid, penicillamine, penicillin V potassium, pentosan polysulfate sodium, phenformin, phenylbutazone, phenyloin sodium, phytoestrogen, potassium chloride, pramipexole, pravastatin sodium, praziquantel, primaquine phosphate, proanthocyanidin, procainamide, promethazine, promethazine hydrochloride, propafenone HCl, propanolol HCl, propoxyphene hydrochloride, propoxyphene napsylate, prozosin, pseudophedrine hydrochloride, pseudoephedrine sulfate, psyllium, pycnogenol, pyrazinamide, pyridostigmine bromide, pyridoxine hydrochloride, pyruvate, quetiapine carafate, quinidine sulfate, quinapril hydrochloride, ramipril, ranitidine hydrochloride, reboxetine, rifabutin, rifampin, risedronate sodium, rofecoxib, rosiglitazone maleate, salbutamol sulfate, saquinavir mesylate, sertraline HCl, sevelamer HCl, sildenafil, simethicone, sodium valproate, sotalol HCl, stavudine, succimer, sumanirole, sumatriptan succinate, suntheanine, terazosin hydrochloride, terbinafine HCl, tetracycline HCl, theophylline, thiobendazole, ticlopidine HCl, timolol meleate, tocamide HCl, tolcapne, tolmetin sodium, tramadol HCl, trovafloxacin mesylate, valacyclovir HCl, valganciclovir HCl, valsartan, vancomycin, venlafaxine hydrochloride, verapamil HCl, warfarin sodium, xylamine, zidovudine, and combinations thereof. Depending on the particular embodiment, the API may be in a solid, powder, particulate, or liquid form.

Suitable flavorants include, but are not limited to menthol, peppermint, mint flavors, fruit flavors, chocolate, vanilla, bubblegum flavors, coffee flavors, liqueur flavors and combinations and the like.

Suitable vitamins and minerals include, but are not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D3; cyanocobalamin; sodium selenite; copper sulfate; vitamin A; vitamin C; inositol; potassium iodide; and combinations thereof. Suitable dosages for vitamins and minerals may be obtained, for example, by consulting the U.S. RDA guidelines. In addition, non-limiting examples of minerals include iron, calcium, magnesium, potassium, copper, chromium, zinc, molybdenum, iodine, boron, selenium, manganese, derivatives thereof or combinations thereof. These vitamins and minerals may be from any source or combination of sources, without limitation. Non-limiting exemplary B vitamins include, without limitation, thiamine, niacinamide, pyridoxine, riboflavin, cyanocobalamin, biotin, pantothenic acid or combinations thereof.

As used herein, the term “nutraceutical” means a natural component of food or other ingestible forms that have been determined to be beneficial to the human body in preventing or treating one or more diseases or improving physiological performance. Essential nutrients can be considered nutraceuticals if they provide a benefit beyond their essential role in normal growth or maintenance of the human body. As used herein, a nutraceutical is defined as any substance that is administered as a dietary supplement to a subject. Such supplements can be purified vitamins or minerals, herbs, or plant extracts. The preferred nutritional supplements are extracts or concentrates of plants, including herbal plants. Examples of the range of such nutritional supplements usable in the invention include, but are not limited to, Chemy extract, Ginkgo biloba extract, Kava Kava extract, Ginseng extract, Saw Palmetto extract, cranberry or blueberry extract, tomato extract, cordyceps sinensis extract, pomegranates, elderberries, as well as the entire berry family, strawberry, raspberry, cherry, black raspberry, boysenberry, etc., glucosamine sulfate, chromium picolinate, Milk thistle extract, Grape seed extract, Ma Huang extract, Co-enzyme Q10.

Suitable plants from which extracts can be prepared and natural substances isolated include but are not limited to the higher plants: Acanthopanax, Acanthopsis, Acanthosicyos, Acanthus, Achyranthes, Acokanthera, Aconitum, Acorus, Acronychia, Actaea, Actinidia, Adenia, Adhatoda, Aegle, Aesculus, Aframomum, Agastache, Agathosma, Alchemilla, Aleurites, Allium, Aloe, Alonsoa, Aloysia, Alphitonia, Alpinia, Alternanthera, Amaranthus, Amomum, Amphipterygium, Amyris, Anchusa, Ancistrocladus, Anemopsis, Angelica, Annona, Anonidium, Anthemis, Antidesma, Apium, Aralia, Aristolochia, Artemisia, Artocarpus, Asarum, Asclepias, Asimina, Aspalanthus, Asparagus, Aspidosperma, Astragalus, Astronium, Atropa, Avena, Azadirachta, Azara, Baccharis, Bacopa, Balanites, Bambusa, Barleria, Barosma, Bauhinia, Belamcanda, Benincasa, Berberis, Berchemia, Bixa, Bocconia, Borago, Boronia, Boswellia, Brosimum, Brucea, Brunfelsia, Bryonia, Buddleja, Bulnesia, Bupleurum, Bursera, Byrsonima, Calamintha, Calea, Calophyllum, Camellia, Camptotheca, Cananga, Canarium, Canella, Capparis, Capsicum, Carthamus, Carum, Cassia, Cassine, Castanospermum, Catalpa, Catha, Catharanthus, Cayaponia, Cecropia, Centaurea, Centipeda, Centranthus, Cephaelis, Chiranthodendron, Chondrodendron, Chrysophyllum, Cimicifuga, Cinchona, Cinnamomum, Cistus, Citrus, Clausena, Cnicus, Coccoloba, Codonopsis, Coffea, Coix, Cola, Coleus, Colletia, Combreturn, Commiphora, Cordia, Coriaria, Correa, Corydalis, Costus, Crataegus, Croton, Cryptolepis, Cudrania, Cuminum, Cuphea, Cucurma, Cyclanthera, Cymbopogon, Cynara, Cynoglossum, Cyperus, Cyrtocarpa, Dalbergia, Dalea, Danae, Daphne, Datura, Daucus, Decadon, Dendrocalamus, Dendropanax, Deppea, Derris, Desmos, Dichrostachys, Dictamnus, Digitalis, Dillenia, Dioscorea, Dioscoreophyllum, Diosma, Diospyros, Drimys, Duboisia, Duguetia, Dysoxylum, Echinacea, Eclipta, Ehretia, Ekebergia, Eleagnus, Elettaria, Eleutherococcus, Encelia, Entandrophragma, Ephedra, Epimedium, Eriobotrya, Erodium, Eryngium, Erythrochiton, Erythroxylum, Escholzia, Esenbeckia, Euclea, Eucommia, Euodia, Eupatorium, Fabiana, Ferula, Fevillea, Fittonia, Flindersia, Foeniculum, Gallesia, Galphimia, Garcinia, Gaudichaudia, Gaultheria, Gelsemium, Gentiana, Geranium, Gigantochloa, Gingko, Glochidion, Gloeospemum, Grewia, Greyia, Guaiacum, Gymnema, Haematoxylum, Hamamelis, Hamelia, Harpagophytum, Hauya, Heimia, Helleborus, Hieracium, Hierochloe, Hilleria, Hippophae, Houttuynia, Hovenia, Humulus, Huperzia, Hura, Hybanthus, Hydnocarpus, Hydnophytum, Hydrastis, Hydrocotyle, Hymenaea, Hyoscamus, Hypericum, Hyptis, Hyssopus, Iboza, Idiospermum, Ilex, Illicium, Indigofera, Inga, Inula, lochroma, Iresine, Iris, Jacaranda, Jatropha, Juniperus, Justicia, Kadsura, Kaempferia, Lactuca, Lagochilus, Larrea, Laurus, Lavandula, Lawsonia, Leonurus, Leucas, Ligusticum, Lindera, Lippia, Liriosma, Litsea, Lobelia, Lonchocarpus, Lonicera, Lycium, Macfadyena, Maclura, Mangifera, Mansoa, Marcgravia, Marrubium, Martinella, Matricaria, Maytenus, Medicago, Melissa, Mentha, Mimosa, Mimusops, Mitragyna, Montanoa, Morkillia, Mouriri, Mucuna, Mutisia, Myrica, Myristica, Nardostachys, Nepeta, Nicotiana, Ocotea, Olea, Oncoba, Ophiopogon, Origanum, Pachyrhizus, Panax, Papaver, Pappea, Parthenium, Passiflora, Paullinia, Pelargonium, Penstemon, Perezia, Perilla, Persea, Petiveria, Petroselinum, Peucedanum, Peumus, Pfaffia, Phoebe, Phyllanthus, Phytolacca, Pilocarpus, Pimenta, Pimpinella, Pinellia, Piper, Piqueria, Pithecellobium, Pittosporum, Plectranthus, Pleuropetalum, Podophyllum, Pogostemon, Polygala, Polygonum, Polymnia, Psacalium, Psychotria, Pterygota, Ptychopetalum, Pueraria, Punica, Pycnanthemum, Pygeum, Quararibea, Quassia, Quillaja, Randia, Ratibida, Rauvolfia, Rehmannia, Renealmia, Rheum, Rollinia, Rorippa, Rosmarinus, Rudbeckia, Ruellia, Rumex, Ruscus, Ruta, Saccharum, Salix, Salvia, Sambucus, Sanguinaria, Sapium, Sassafras, Satureja, Sceletium, Schizandra, Securidaca, Securinega, Serenoa, Simmondsia, Smilax, Stachytarpheta, Stachys, Staurogyne, Stelechocarpus, Stephania, Sterculia, Stevia, Strophanthus, Strychnos, Symphytum, Syzygium, Tabebuia, Tabemaemontana, Tabemanthe, Tanacetum, Taxus, Tecoma, Terminalia, Teucrium, Thaumatococcus, Tribulus, Trifolium, Trigonella, Triplaris, Triumfetta, Tumera, Tussilago, Tylophora, Tynnanthus, Uncaria, Urginea, Urtica, Uvaria, Vaccinium, Valeriana, Vallesia, Vangueria, Vanilla, Vellozia, Vepris, Verbascum, Verbena, Vetiveria, Virola, Viscum, Vismia, Vitex, Voacanga, Warburgia, Withania, Zanthoxylum, Zingiber, Zizyphus and Zygophyllum. In addition to the genera of higher plants listed above, compounds can be recovered from such biological sources as algae, bacteria, fungi, lichens, mosses, and marine organisms such as corals, sponges, tunicates or other invertebrate or vertebrate organisms.

As used herein, the term “fatty acid” is art recognized and includes a long-chain hydrocarbon based carboxylic acid. Fatty acids are components of many lipids including glycerides. The most common naturally occurring fatty acids are monocarboxylic acids which have an even number of carbon atoms (16 or 18) and which may be saturated or unsaturated. “Unsaturated” fatty acids contain cis double bonds between the carbon atoms. Suitable unsaturated fatty acids include, but are not limited to unsaturated fatty acids can include, but are not limited to, gamma linolenic acid (GLA), alpha linolenic acid (ALA), stearidonic acid (SDA), arachidonic acid (AA), eicosapentaenoic acid (EPA), docosapentaenoic acid (DPA), docosahexaenoic acid (DHA), and linoleic acid (LA).

Various other pharmaceutically acceptable excipients may be included in the translucent semi-solid fill material, such as preservatives, e.g., methyl- or propylparaben, coloring agents, flavoring agents, lubricants, flow-enhancers, anti-oxidants, surfactants, plasticizers, filling aids and other compounds, agents and components which produce an appealing final product.

The active ingredient or ingredients are generally present in the core in a therapeutically effective amount, which is an amount that produces the desired therapeutic response upon oral administration and can be readily determined by one skilled in the art. In determining such amounts, the particular active ingredient being administered, the bioavailability characteristics of the active ingredient, the dosing regimen, the age and weight of the patient, and other factors must be considered, as known in the art. In one embodiment, the dosage form comprises at least about 0.1 weight percent (based on the weight of the core) of one or more active ingredients.

The active ingredient or ingredients may be present in the dosage form in any form. For example, one or more active ingredients may be dispersed at the molecular level, e.g. melted or dissolved, within the dosage form, or may be in the form of particles, which in turn may be coated or uncoated. If an active ingredient is in form of particles, the particles (whether coated or uncoated) typically have an average particle size of about 1-2000 microns. In one preferred embodiment, such particles are crystals having an average particle size of about 1-300 microns. In another preferred embodiment, the particles are granules or pellets having an average particle size of about 50-2000 microns, preferably about 50-1000 microns, most preferably about 100-800 microns.

In certain embodiments, at least a portion of one or more active ingredients may be optionally coated with a release modifying coating, as known in the art. This advantageously provides an additional tool for modifying the release profile of active ingredient from the dosage form. For example, the core may contain coated particles of one or more active ingredients, in which the particle coating confers a release modifying function, as is well known in the art. Examples of suitable release modifying coatings for particles are described in U.S. Pat. Nos. 4,173,626; 4,863,742; 4,980,170; 4,984,240; 5,86,497; 5,912,013; 6,270,805; and 6,322,819. Commercially available modified release coated active particles may also be employed. Accordingly, all or a portion of one or more active ingredients in the core may be coated with a release-modifying material.

In embodiments in which it is desired for at least one active ingredient to be absorbed into the systemic circulation of an animal, the active ingredient or ingredients are preferably capable of dissolution upon contact with a dissolution medium such as water, gastric fluid, intestinal fluid or the like.

In one embodiment, the dissolution characteristics of at least one active ingredient meets USP specifications for an immediate release formulation containing the active ingredient. One skilled in the art will appreciate the specific USP specifications will vary depending on the active ingredient selected.

In another embodiment, the dissolution characteristics of one or more active ingredients are modified: e.g. controlled, sustained, extended, retarded, prolonged, delayed and the like. In one embodiment in which one or more active ingredients are released in a modified manner, the modified release active or actives are preferably contained in the core. As used herein, the term “modified release” means the release of an active ingredient from a composition or a portion thereof in other than an immediate release fashion, i.e., other than immediately upon contact of the composition or portion thereof with a liquid medium. As known in the art, types of modified release include delayed or controlled. Types of controlled release include prolonged, sustained, extended, retarded, and the like. Modified release profiles that incorporate a delayed release feature include pulsatile, repeat action, and the like. As is also known in the art, suitable mechanisms for achieving modified release of an active ingredient include diffusion, erosion, surface area control via geometry and/or impermeable or semi-permeable barriers, and other known mechanisms.

In addition to the core, the gelatin compositions of the currently claimed embodiments also generally comprise an outer surface coating. The outer surface coating comprises a rapid-release agent and a gelatin component, and is typically applied to the distal surface of the core. The outer surface coating is exposed to the external environment and is typically designed to provide rapid dissolution once in contact with gastric or intestinal fluid, thereby exposing all or a portion of the core to the gastric or intestinal fluid.

The outer surface coating may generally be utilized as a component of a coated tablet, soft gel-caps, and hard capsules. Other uses of the encapsulation composition embodiments may include, but are not limited to, chewable compositions and chewing gum that includes an encapsulated active therapeutic compound.

In order to inhibit the formation of cross-bridges within the encapsulation during storage at conditions including but not limited to elevated temperature, elevated humidity, and combinations thereof, the gelatin compositions may further include hydrolysates of gelatin. Because the formation of cross-bridges in the encapsulation may hamper the dissolution of the encapsulation, the addition of gelatin hydrolysates may maintain the initial dissolution characteristics of the gelatin composition, even after extended storage periods. In an exemplary embodiment, the hydrolysates of gelatin included in the gelatin capsule composition have molecular weights ranging from about 100 to about 2000 Daltons.

The outer surface coating of the currently claimed embodiments generally incorporate a rapid-release agent and a gelatin component. The rapid-release agent generally functions by releasing a gas including, but not limited to, carbon dioxide, as a by-product of the dissociation of the rapid-release agent upon contact with gastric or intestinal fluids. Without being bound to any particular theory, the dissociation of the rapid-release agent releases gas bubbles within the outer surface coating. The hydrostatic pressure of the gas bubbles released by the dissociation of the rapid-release agent in the encapsulation exerts physical stresses on the surrounding area, causing the tearing and ultimate rupture of the coating. The disruptive forces of the gas bubbles released by the dissociation of the rapid-release agent induces a significantly more rapid release of an active compound encapsulated by the outer surface coating compared to a composition that lacks the rapid-release agent.

The outer surface coating may include a thickness that varies depending on the desired properties of the formulation. In one embodiment, the thickness of the outer surface coating may range from about 0.01 mm to about 10 mm, from about 0.1 mm to about 5 mm, and from about 0.5 mm to about 3 mm. Specifically, the thickness of the outer surface coating may include about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, and about 2.0 mm.

Suitable rapid-release agents may include, but are not limited to water-soluble and water-insoluble bicarbonate and carbonate salts. Water-soluble rapid-release agents may be selected to be any compounds that are at least partially soluble in water, in addition to being soluble in an aqueous solution at a pH ranging from 0 to about 3. The term “at least partially soluble” generally includes compounds with a solubility in water of at least 1% (1 gram in 100 ml of water at 20° C.). Suitable water-soluble rapid-release agents include, but are not limited to, sodium carbonate, potassium carbonate, ammonium carbonate, sodium bicarbonate, potassium bicarbonate, ammonium bicarbonate, calcium bicarbonate, and combinations thereof.

In another embodiment, the rapid-release agent may include water-insoluble carbonate salts. Suitable water-insoluble rapid-release agents may be selected to be any compounds that are essentially insoluble in an aqueous solution at a pH ranging from about 6 to about 8, in addition to being soluble in an aqueous solution at a pH ranging from 0 to about 3. As used herein, the term “essentially insoluble” is indicative of a solubility in water of less than 1% (1 g in 100 ml of water at 20° C.). Suitable water-insoluble rapid-release agents include, but are not limited to, bismuth subcarbonate, calcium carbonate, cobalt carbonate, lanthanum carbonate, lead carbonate, lithium carbonate, magnesium carbonate, manganese carbonate, nickel (II) carbonate, silver carbonate, strontium carbonate, and combinations thereof. In one particular embodiment, the water-insoluble rapid-release agent may be calcium carbonate.

For various edible gelatin compositions including but not limited to foods, dietary supplements, and pharmaceutical products, the rapid-release agents may be of at least food grade quality. More preferably, the rapid-release agents may be of GRAS and USP quality.

The rapid-release agent may be utilized in the form of fine particles less than about 0.152 mm (about 100 mesh) in size. The fine particles may be less than about 0.089 mm (about 170 mesh), less than about 0.075 mm (about 200 mesh), less than about 0.066 mm (about 230 mesh), or less than about 0.053 mm (about 270 mesh) in size. In another embodiment, the rapid-release agents may be utilized in the form of fine particles less than about 0.075 mm (about 200 mesh) in size.

The amount of rapid-release agent included in the gelatin capsule compositions may be sufficiently high to induce the formation of gas bubbles such as carbon dioxide when the gelatin composition is exposed to an acidic solution such as gastric juices. Rapid-release agent may be included in the gelatin composition in an amount ranging from about 0.1% to about 50% of the weight of the gelatin component. Alternatively, the amount of rapid-release agent included in the gelatin capsule compositions may range from about 5% to about 13%, from about 9% to about 17%, from about 10% to about 18%, from about 14% to about 22%, from about 18% to about 26%, from about 22% to about 30%, from about 26% to about 34%, from about 30% to about 36%, from about 34% to about 40%, from about 38% to about 44%, from about 42% to about 48%, and from about 46% to about 50% of the total weight of the composition. In one embodiment, the amount of rapid release agent included in the gelatin capsule compositions comprises about 1% to about 20% based on the weight of the gelatin. In another embodiment, the amount of rapid release agent included in the gelatin compositions comprises about 10% to about 30% based on the weight of the gelatin. In a further embodiment, the amount of rapid release agent included in the gelatin capsule compositions comprises about 15% to about 20% based on the weight of the gelatin.

Preferably, the amount of rapid-release agent included in the gelatin capsule composition may be sufficient to induce the formation of bubbles when the composition is contacted with an acidic solution such as gastric juices. Higher proportions of rapid-release agents such as calcium carbonate may result in a gelatin composition with undesirably brittle material properties.

In addition to the rapid-release agent, the gelatin capsule compositions also include a gelatin component. The gelatin component may be derived from collagen or collagen rich tissue including, but not limited to, the skin and bones of pigs or cattle. Non-limiting examples of gelatin include Type A gelatin, Type B gelatin and combinations thereof. Type A gelatin is characterized by an isoionic point ranging from about 7 to about 10.0, and is typically derived from collagen using an acid pretreatment method known in the art. Type B gelatin is characterized by an isoionic point ranging from about 4.8 to about 5.8.

The gelatin may typically include from about 80% to about 90% by weight protein, from about 0.1% to about 2% by weight mineral salts and from about 10% to 15% by weight water. The term “protein”, as defined herein, refers to organic compounds made up of a plurality of amino acids joined together by peptide bonds between the carboxyl and amino groups of each adjacent amino acid. The gelatin may have an average molecular weight ranging from about 50,000 Da to about 300,000 Da. In another embodiment, the gelatin has an average molecular weight ranging from about 70,000 Da to about 150,000 Da. In a further embodiment, the gelatin has an average molecular weight ranging from about 80,000 Da to about 120,000 Da. Additionally, the gelatin typically comprises a Bloom value from about 50 to about 300. In one embodiment, the gelatin comprises a Bloom value ranging from about 125 to about 200. In yet another embodiment, the Bloom value may range from about 150 to about 175. The gelatin typically has a pH from about 3.8 to about 7.5. In another embodiment, the gelatin has a pH ranging from about 6.2 to about 7.3. In a further embodiment, the gelatin comprises a pH ranging from about 6.6 to about 7.0. The gelatin may also comprise an isoelectric point from about 4.7 to about 9.0, a viscosity from about 15 to about 75 mPas and the ash content ranging from about 0.1% to about 2.0% by weight.

If the gelatin is substantially Type A gelatin, the bloom strength may range from about 50 to about 300, the pH may generally range from about 3.8 to about 5.5, the isoelectric point may range from about 7.0 to about 9.0, the viscosity may range from about 15 to about 75 mPas and the ash content may range from about 0.1% to about 2.0% by weight.

If the gelatin is substantially Type B gelatin, the bloom strength may generally range from about 50 to about 300, the pH may generally range from about 5.0 to about 7.5, the isoelectric point may range from about 4.7 to about 5.8, the viscosity may range from about 20 to about 75 mPas and the ash content may range from about 0.5% to about 2.0% by weight.

The gelatin may optionally be deionized prior to use by known methods including, but not limited to, ion exchange using a mixed bed of ion-exchange resin. The gelatin may also include gelatin hydrolysates having molecular weights ranging from about 100 Da to about 2000 Da. The gelatin hydrolysates and methods of producing the hydrolysates are described in U.S. Pat. No. 7,485,323, which is hereby incorporated by reference in its entirety.

The physical properties of the gelatin can and will vary depending upon its intended use. In one embodiment wherein the gelatin is used in the manufacture of hard capsule pharmaceutical products, the gelatin may have a bloom strength ranging from about 200 to about 300, a viscosity ranging from about 40 to about 60 mPas and a pH ranging from about 4.5 to about 6.5. In another embodiment wherein the gelatin is used in the manufacture of soft shell capsule pharmaceutical products, the gelatin may have a bloom strength ranging from about 125 to about 200, a viscosity ranging from about 25 to about 45 mPas a pH ranging from about 4.5 to about 6.5.

The particular gelatin of the gelatin capsule composition may be selected to possess an isoelectric point of below about 7.5. Because the addition of rapid-release agents may result in a composition pH that is significantly higher than previous gelatin encapsulation compositions, lower gelatin isoelectric points reduce the likelihood of occurrence of adverse chemical processes including but not limited to gelatin deamidation during the production of the gelatin capsule composition.

The viscosity of the gelatin suspension used to produce the gelatin capsule composition may be at a level at which the rapid release agents may not remain suspended during production. In these cases, an additional thixotropic compound including but not limited to a hydrocolloid such as hydroxyethyl cellulose or carboxymethyl cellulose may be added to the gelatin capsule composition in order to maintain a sufficiently high viscosity of the gelatin suspension during production of the gelatin capsule composition.

In addition, the term gelatin or gelatin component may be interpreted to encompass combinations of gelatin and other formulation additives such as plasticizers, aqueous solvents or mediums, and other components known in the art. The term plasticizer (also known as a dispersant) is generally used to describe additives that impart increased flexibility and pliability to the gelatin component. Specifically, the plasticizer may be hydrophilic such as triethyl citrate and polyethylene glycol and/or hydrophobic such as diethyl phthalate, dibutyl phthalate, dibutyl sebacate and acetyl tributyl citrate. One skilled in the art will understand that other materials may be substituted for the polymer/plasticizer if they are capable of fulfilling the same function, i.e. imparting an increased level of flexibility and pliability to the gelatin formulation. It is possible that various hydrophobic materials including oils and waxes may also be used in this regard and can be found through routine experimentation or in the literature known to the skilled artisan. In one embodiment, the plasticizer comprises dibutyl sebacate, diethyl phthalate, glycerine, polyethylene glycol, propylene glycol, sorbitol, sorbitans, triacetin, triethyl citrate, water, and mixtures thereof. In another embodiment, the plasticizer is selected from the group consisting of glycerine, sorbitol, erythritol, and combinations thereof. One skilled in the art will appreciate that the various plasticizer compounds discussed herein and known within the art may encompass any of the commercially available products incorporating the plasticizers discussed herein. In one embodiment, the plasticizer may comprise a combination of sorbitol and one or more sorbitans, as marketed under the tradename POLYSORB® sold by Roquette.

In one embodiment, the plasticizer comprises a mixture of glycerine and sorbitol in a weight ratio ranging from 10:1 to 1:10, from 6:1 to 1:6, from 3:1 to 1:3, and a weight ratio of about 1:1. In yet another embodiment, the plasticizer comprises the combination of a mixture of sorbitol and sorbitans, glycerine, and water. The combination of the sorbitol and sorbitans mixture, glycerine, and water may generally comprise about 5 to about 95% sorbitol and sorbitans mixture based on the weight of the plasticizer component, about 5% to about 95% glycerine based on the weight of the plasticizer component, and about 1% to about 25% water based on the weight of the plasticizer component. In one embodiment, the plasticizer comprises about 40% to about 60% sorbitol and sorbitans mixture based on the weight of the plasticizer component, about 35% to about 50% glycerine based on the weight of the plasticizer component, and about 2% to about 12% water based on the weight of the plasticizer component. In still another embodiment, the combination of sorbitol, glycerine, and water comprises about 50% sorbitol based on the weight of the plasticizer component, about 42.5% glycerine based on the weight of the plasticizer component, and about 7.5% water based on the weight of the plasticizer component. In still another embodiment, the plasticizer comprises a combination of 50% POLYSORB® and 50% of a glycerine solution comprising 85% glycerine and 15% water.

The term aqueous solvent or aqueous medium may be interpreted to encompass any solvent capable of forming a gelatin composition. The aqueous medium may include, but is not limited to water.

In addition, the currently claim embodiments may further include a pH-altering compound that increases or decreases the pH of the gelatin component. In one embodiment, the gelatin composition may also include sodium hydroxide to increase the pH of the composition. One skilled in the art will appreciate that other basic or acidic compounds may be added to develop compositions with specific pH values, thereby affecting the dissolution profile of the rapid-release agent.

In one embodiment, the gelatin component of the gelatin capsule composition comprises a combination of gelatin, a plasticizer, and an aqueous medium comprising water. The gelatin component may include about 25% to about 55% by weight of the gelatin component of the gelatin, about 15% to about 30% by weight of the gelatin component of the plasticizer, and about 25% to about 40% by weight of the gelatin component of the water. In a further embodiment, the gelatin component comprises about 35% to about 45% by weight of the gelatin component of the gelatin, about 20% to about 25% by weight of the gelatin component of the plasticizer, and about 30% to about 35% by weight of the gelatin component of the water. In yet another embodiment, the gelatin component comprises about 40% by weight of the gelatin component of the gelatin, about 20% by weight of the gelatin component of the plasticizer, and about 23% by weight of the gelatin component of the water.

In an additional embodiment, the gelatin component of the gelatin capsule composition comprises a combination of gelatin and an aqueous medium comprising water. The gelatin component may include from about 5% to about 30% gelatin by weight of the gelatin component and from about 70% to about 95% water by weight of the gelatin component. In another embodiment, the gelatin component comprises from about 10% to about 20% gelatin by weight of the gelatin component and from about 80% to about 90% water by weight of the gelatin component.

In another embodiment, the gelatin component may also include gelatin hydrolysates having molecular weights ranging from about 100 Da to about 2000 Da. The weight ratio of the gelatin to the gelatin hydrolysate generally ranges from about 3:1 to about 99:1. In another embodiment, the weight ratio of the gelatin to the gelatin hydrolysate ranges from about 4:1 to about 49:1. In yet another embodiment, the weight ratio of the gelatin to the gelatin hydrolysate ranges from about 5:1 to about 19:1.

In one aspect of the currently claimed embodiments, the outer surface coating incorporates one or more first distinct regions comprising the gelatin component and one or more second distinct regions comprising the rapid-release agent. The one or more second distinct regions comprising a rapid-release agent may generally span from the distal surface of the outer surface coating that is exposed to the external environment to the proximal surface of the outer surface coating in contact with the core. The one or more second distinct regions comprising the rapid-release agent may generally be described as functional stripes, bars, bands, streaks, strips, rows, columns, spots, flecks, striations, belts, ribbons, veins, dashes, ridges, strains, etc., that are present on the distal surface of the outer surface coating, and penetrate the gelatin layer toward the proximal surface. It should be understood that the one or more second regions may span the entire depth of the outer surface coating, from the distal surface to the proximal surface, or may span only a portion of the depth of the outer surface coating extending from the distal surface to the proximal surface. In one embodiment, the one or more second distinct regions comprising a rapid-release agent penetrates the entire width of the outer surface coating surrounding the core, such that the rapid dissolution of the rapid-release agent allows for the emptying of the contents of the core to the exterior environment, without having to wait for the entire gelatin mass of the outer surface coating to dissolve.

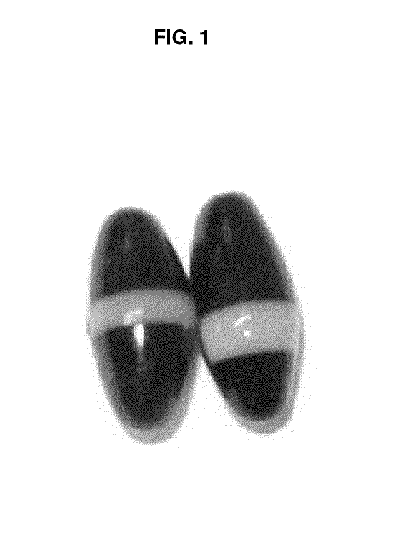

The one or more second distinct regions may be uniformly spread throughout the gelatin capsule (i.e., stripes or bands of uniform width, shape, etc) or may comprise a non-uniform pattern throughout (i.e., stripes or bands with no uniform width, shape, etc.), generally resembling a marbled look on the outer surface coating. In one embodiment, as illustrated in FIG. 1, the gelatin composition may include one distinct region comprising the rapid-release agent (shown in white) and two distinct regions comprising the gelatin component without any rapid-release agent present, wherein the one distinct region comprising a rapid-release agent is of uniform size and shape. As shown in FIG. 1, the distinct regions may vary in width and appearance.

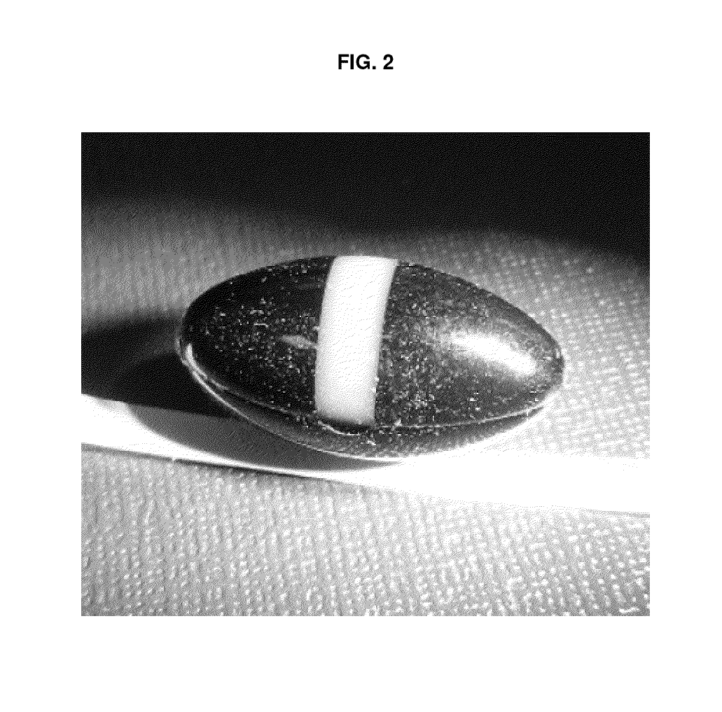

In a further embodiment, as illustrated in FIG. 2, the gelatin composition may comprise a second distinct region that only extends over a portion of the circumference of the gelatin composition. In the embodiment illustrated in FIG. 2, the distinct region comprising a rapid-release agent (shown in white) extends over approximately one half of the circumference of the gelatin composition, creating one half of the composition comprising two distinct regions composed of the gelatin component and one distinct region composed of the rapid release agent, and a second half composed entirely of the gelatin component. One skilled in the art will appreciate that the size, shape, orientation, etc. may be customized to provide for novel release profiles, depending on the needs of the manufacturer.

The width of the one or more second distinct regions may range from about 0.01 mm to about 5 mm. Specifically, the thickness of the outer surface coating may include about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, and about 2.0 mm.

Generally, the one or more second distinct regions provide a mechanism by which the gelatin capsule can release the contents of the core more quickly than other dosage forms. Specifically, upon exposure to an aqueous, acidic environment, the rapid-release agent dissolves in the medium, releasing a gas. Because the rapid-release agent is concentrated in distinct regions that span at least a portion of the distance from the distal surface to the proximal surface of the outer surface coating, the rapid dissolution creates tears and splits in the gelatin capsule, leading to the rapid release of the contents of the core into the surrounding environment. Unlike other formulations, wherein the rapid-release agent is dispersed throughout the core formulation (in combination with the active ingredient and other excipients), the gelatin compositions of the current claimed embodiments rely on rapid degradation of the outer surface coating at the one or more second distinct regions, resulting in the rapid tearing and breaking of the surface of the gelatin capsule.

In one embodiment, both the one or more first distinct regions and the one or more second distinct regions may comprise a gelatin component, but only the one or more second distinct regions includes a rapid-release agent. The relative amount of gelatin component between the first region(s) and the second region(s) may vary depending upon the desired qualities; however, the skilled artisan will appreciate that the one or more second distinct regions may incorporate more, less, or equal amounts of the gelatin component compared to the one or more first regions. In an alternative embodiment, both the one or more first distinct regions and the one or more second distinct regions incorporate both a gelatin component and a rapid-release agent; however, in such an embodiment, the concentration of the rapid-release agent in the one or more first distinct regions is generally less than the concentration of the rapid-release agent present in the one or more second distinct regions so as to provide a breaking or tearing of the gelatin composition when the one or more second distinct regions is exposed to gastric or intestinal fluid.

The one or more first distinct regions and the one or more second distinct regions may comprise a gelatin component including, but not limited to gelatin, plasticizers, and aqueous mediums comprising water. Specifically, the gelatin component of the one or more first distinct regions comprise about 25% to about 55% by weight of the one or more first distinct regions of the gelatin, about 10% to about 30% by weight of the one or more first distinct regions of the plasticizer, and about 15% to about 45% by weight of the one or more first distinct regions of water. In another embodiment, the gelatin component of the one or more first distinct regions comprise about 35% to about 45% by weight of the one or more first distinct regions of the gelatin, about 16% to about 24% by weight of the one or more first distinct regions of the plasticizer, and about 20% to about 30% by weight of the one or more first distinct regions of the water. In a further embodiment, the gelatin component of the one or more first distinct regions comprises about 40% gelatin, about 20% plasticizer, and about 23% water.

In yet another embodiment, the one or more second distinct regions include the combination of gelatin, a rapid-release agent, a plasticizer, and water. The one or more second distinct regions may comprise about 20% to about 50% by weight of the one or more second distinct regions of the gelatin, about 1% to about 25% by weight of the one or more second distinct regions of the rapid-release agent, about 10% to about 30% by weight of the one or more second distinct regions of the plasticizer, and about 20% to about 40% by weight of the one or more second distinct regions of water. In still another embodiment, the one or more second distinct regions comprise about 30% to about 45% by weight of the one or more second distinct regions of the gelatin, about 4% to about 16% by weight of the one or more second distinct regions of the rapid-release agent, about 16% to about 24% by weight of the one or more second distinct regions of the plasticizer, and about 26% to about 34% by weight of the one or more second distinct regions of water. In a further embodiment, the one or more second distinct regions include about 37% by weight of the one or more second distinct regions of gelatin, about 7.75% by weight of the one or more second distinct regions of one or more rapid-release agents, about 20% by weight of the one or more second distinct regions of plasticizer, and about 30% by weight of the one or more second distinct regions of water.

The one or more first or second distinct regions, as described herein, may further incorporate a gelatin hydrolysate as previously described. The gelatin hydrolysate helps to prevent the development of pellicles (insoluble gelatin formations), which tend to hinder release of the contents of the core, affecting the efficacy of the gelatin composition. Incorporation of the gelatin hydrolysate into the one or more first distinct regions will improve the solubility of the region, decreasing the time required to solubilize the gelatin capsule. Similarly, incorporation of the gelatin hydrolysate into the one or more second distinct regions provides a base that rapidly dissolves once the rapid-release agent comprising the one or more second distinct regions is exposed to an aqueous, acidic environment. Generally, the gelatin hydrolysate may be incorporated into the gelatin component in concentrations ranging from about 0.1% to about 20% by weight of the one or more first distinct regions or the one or more second distinct regions. In one embodiment, the gelatin hydrolysate may be incorporated into the gelatin component in an amount ranging from about 1% to about 15% by weight of the one or more first distinct regions or the one or more second distinct regions. In still another embodiment, the gelatin hydrolysate may be incorporated into the gelatin component in an amount ranging from about 5% to about 10% by weight of the one or more first distinct regions or the one or more second distinct regions.

In certain embodiments wherein the gelatin hydrolysate is incorporated into the formulation, the one or more first distinct regions may include about 25% to about 55% by weight of the one or more first distinct regions of the gelatin, about 0.01% to about 20% by weight of the one or more first distinct regions of the gelatin hydrolysate, about 10% to about 30% by weight of the one or more first distinct regions of the plasticizer, and about 15% to about 45% by weight of the one or more first distinct regions of water. In another embodiment, the one or more first distinct regions comprise about 35% to about 48% by weight of the one or more first distinct regions of the gelatin, about 1% to about 10% by weight of the one or more first distinct regions of the gelatin hydrolysate, about 16% to about 24% by weight of the one or more first distinct regions of the plasticizer, and about 20% to about 30% by weight of the one or more first distinct regions of water. In an additional embodiment, the one or more first distinct regions include about 40% gelatin, about 5% gelatin hydrolysate, about 20% plasticizer, and about 23% water.

Additionally, the one or more second distinct regions may include about 20% to about 50% by weight of the one or more second distinct regions of the gelatin, about 0.01% to about 20% by weight of the one or more second distinct regions of the gelatin hydrolysate, about 1% to about 25% by weight of the one or more second distinct regions of the rapid-release agent, about 10% to about 30% by weight of the one or more second distinct regions of the plasticizer, and about 20% to about 40% by weight of the one or more second distinct regions of water. In a further embodiment, the one or more second distinct regions may include about 30% to about 45% by weight of the one or more second distinct regions of the gelatin, about 1% to about 10% by weight of the one or more second distinct regions of the gelatin hydrolysate, about 4% to about 16% by weight of the one or more second distinct regions of the rapid-release agent, about 16% to about 24% by weight of the one or more second distinct regions of the plasticizer, and about 26% to about 34% by weight of the one or more second distinct regions of water. In still another embodiment, the one or more second distinct regions include about 37% gelatin, about 5% gelatin hydrolysate, about 7.75% rapid-release agent, about 20% plasticizer, and about 30% water.

One of skill in the art will appreciate that the gelatin and gelatin hydrolysate components of the pharmaceutical composition will dissolve in the absence of acidic conditions; however, the rapid-release agent generally will not dissociate and release gas, unless exposed to acidic conditions. Thus, the release of the contents of the core is greatest when the proposed pharmaceutical composition is exposed to an acidic environment.

The gelatin compositions with one or more first distinct regions comprising a gelatin component and one or more second distinct regions comprising a rapid-release agent may be produced by any method known in the art capable of producing the desired product. In one embodiment, the components of the one or more first distinct regions and the one or more second distinct regions may be separately fed through a static mixer to produce the desired stripes, bands, etc. Additionally, the gelatin capsules incorporating the active pharmaceutical ingredient may be produced according to any method known within the art, including extrusion processes.

In another embodiment, the outer surface coating may comprise a homogenous or semi-homogenous distribution of the rapid-release agent throughout the gelatin components, such that there are not distinct regions comprising the rapid-release agent and distinct regions comprising the gelatin component. In this embodiment, upon exposure to an aqueous, acidic environment the entire surface of the outer surface coating degrades, such that there are not distinct regions degrading at an accelerated rate compared to the remainder of the coating, releasing the contents of the core into the environment for absorption. Upon exposure to an acidic environment, the rapid-release agent particles will dissociate from the gelatin, causing the release of gas. In addition to the dissolution of the gelatin, the added release of gas caused by rapid-release agent results in more rapid degradation of the gelatin and release of the core contents. Because the gelatin composition degrades across the entire surface of the capsule, the degradation results in the eventual breakage of gelatin surface.

In an additional aspect, the currently claimed embodiments provide a method for manufacturing the gelatin compositions comprising a gelatin component and a rapid-release agent. The gelatin composition comprising a rapid release agent and a gelatin component may be manufactured according to the following steps: (a) dissolving a gelatin component in an aqueous medium; (b) mixing a rapid-release agent with the aqueous gelatin solution prior to capsule formation; and (c) incorporating the combination of the aqueous gelatin solution and rapid-release agent to form an outer surface coating that is applied to a core. Step (a) comprising dissolving a gelatin component in an aqueous medium may be carried out by dissolving the gelatin in an aqueous medium by any process known in the art to form an aqueous solution ranging from about 5% to about 60% gelatin by weight. In other embodiments, the aqueous gelatin solution may range from about 5% to about 15%, from about 10% to about 20%, from about 15% to about 25%, from about 20% to about 30%, from about 25% to about 35%, from about 30% to about 40%, from about 35% to about 45%, from about 40% to about 50%, from about 45% to about 55%, or about 50% to about 60% gelatin by weight. In particular, the aqueous solution may be about 15% gelatin by weight.

The gelatin may have varying particle sizes prior to the addition of the gelatin to the water to form the aqueous solution. In one embodiment, the gelatin particle sizes may vary from about 0.1 mm to about 10 mm. In other embodiments, the gelatin particle size may range from about 0.1 to about 0.3 mm, from about 0.2 to about 0.8 mm, from about 0.5 to about 1.5 mm, from about 1 to about 3 mm, from about 2 to about 6 mm, or from about 5 to about 10 mm. Without being bound to any particular theory, the particle size of the gelatin may impact the amount of time needed for the gelatin to degrade in aqueous solution. Gelatins having a particle size ranging from about 0.1 to about 0.3 mm may swell in solution within a few minutes, gelatins having a particle size ranging from about 0.3 to about 0.8 mm may swell in solution within a time from about 8 to about 12 minutes, and gelatins having a particle size greater than about 0.8 mm may swell within about an hour.

Gelatin solutions having a concentration ranging from about 10% to about 20% by weight of gelatin may be prepared using any gelatin particle size. In another embodiment having a more concentrated solution ranging from about 30% to about 34% gelatin by weight, gelatin particles larger than about 0.8 mm in size may be used to inhibit aggregation and air bubble formation during processing.

In one embodiment, either during step (a) or immediately after, the pH of the aqueous gelatin solution may be adjusted to a pH ranging from about 6 to about 11 by the addition of an acid or base. In another embodiment, the gelatin solution is adjusted to a pH level ranging from about 8 to about 10 prior to step (b). In a further embodiment, the pH of the gelatin solution is adjusted to about 8.9, prior to step (b). In those embodiments in which the gelatin composition is to be used for pharmaceutical encapsulant applications, suitable acids may include food grade acids. Non-limiting examples of suitable food-grade acids include sulfuric acid, tartaric acid, citric acid, acetic acid, and carbon dioxide gas from carbon dioxide sources including, but not limited to, dry ice, phosphoric acid, or combinations thereof. Non-limiting examples of suitable food-grade bases include sodium hydroxide, potassium hydroxide, calcium hydroxide, sodium bicarbonate, potassium bicarbonate, calcium oxide or combinations thereof. Without being bound to any particular theory, the adjustment of the pH may alter the cross-linking or ionic interactions of gelatin molecules, thereby altering the material properties of the resulting gelatin encapsulant material including, but not limited to, hardness, solubility in aqueous solution having a low pH ranging from 0 to about 3, and combinations thereof.

Step (b) of the process is directed to mixing the rapid-release agent with the aqueous solution of gelatin prior to gelatin coating or capsule formation. The mixing process forms an outer surface coating composition, wherein the composition generally comprises a mass ratio of the rapid-release agent to the gelatin ranging from about 1:1 to about 1:99. The mass ratio is defined as the ratio of the mass of rapid-release agent compared to the mass of the gelatin solution, wherein the gelatin solution comprises the combined mass of the gelatin, the aqueous solution in which the gelatin is dissolved, as well as the mass of any gelatin hydrolysate incorporated into the solution. Alternatively, the mass ratio of the rapid-release agent to the gelatin may range from about 1:1 to about 1:8, from about 1:4 to about 1:10, from about 1:6 to about 1:12, from about 1:8 to about 1:14, from about 1:10 to about 1:16, from about 1:12 to about 1:18, from about 1:14 to about 1:20, from about 1:16 to about 1:22, from about 1:18 to about 1:24, from about 1:20 to about 1:26, from about 1:22 to about 1:28, from about 1:24 to about 1:30, from about 1:26 to about 1:32; from about 1:28 to about 1:34, from about 1:30 to about 1:36, from about 1:32 to about 1:38, from about 1:34 to about 1:40, from about 1:36 to about 1:42, from about 1:38 to about 1:44, from about 1:40 to about 1:46, from about 1:42 to about 1:48, and from about 1:44 to about 1:50 in the encapsulation composition.

In one embodiment, the mass ratio of the rapid-release agent to the gelatin may range from about 1:2 to about 1:19 in the gelatin composition. In a further embodiment, the mass ratio of the rapid-release agent to the gelatin may range from about 1:4 to about 1:9 in the gelatin composition.

In one embodiment, the rapid-release agent is mixed with the aqueous gelatin solution prior to or during step (c). The period of time prior to step (c) may range from immediately prior to hours before step (c) is executed. In some embodiments, the rapid-release agent may alter the pH of the gelatin solution slightly but the magnitude of the change may not be sufficient to cause undue instability of the gelatin to hydrolysis.

Further, the mixing process associated with step (b) may include any process known within the art that allows for the mixing of the rapid-release agent prior to or during the incorporation of the aqueous gelatin solution into the coating or capsule forming machine. In one embodiment, step (b) may be accomplished by mixing the rapid-release agent with the aqueous gelatin solution by means of an in-line mixing process. In another embodiment, the in-line mixing process of step (b) is accomplished by means of a static mixer. A static mixer generally consists of a series of alternating left and right hand helical elements. Non-limiting examples of static mixers include, but are not limited to kenics static mixers. The in-line mixing process allows for the rapid-release agent and the aqueous gelatin solution to be simultaneously introduced into the in-line mixer, whereby immediately after mixing, the combination of the rapid-release agent and the aqueous gelatin solution are fed into a coating or capsule producing machine or apparatus. Mixing the two components together immediately prior to or during incorporation of the combination into the coating or capsule forming process, decreases the amount of time available for the rapid-release agent to raise the pH of the aqueous gelatin solution. By minimizing the amount of time that the water-soluble rapid-release agent and the aqueous gelatin solution are mixed, the likelihood that the viscosity of aqueous gelatin solution will be decreased is minimized. Thus, step (b) of the presently claimed embodiment avoids the solution stability issues present when the water-soluble rapid-release agent and aqueous gelatin solution are combined for significant amounts of time prior to coating or capsule formulation.

Step (c) of the presently claimed embodiment comprises incorporating or feeding the combination of the water-soluble rapid-release agent and the aqueous gelatin solution into a coating or capsule forming machine. It will be understood by one skilled in the art that a variety of machines may be utilized to produce the rapid-release encapsulation composition.

In one embodiment, the gelatin composition includes gelatin in an aqueous solution containing about 15% gelatin by weight, and from about 2% to about 10% sodium bicarbonate by weight. Another exemplary gelatin capsule composition includes gelatin in aqueous solution containing about 15% gelatin by weight, and from about 5% to about 15% calcium carbonate by weight. The particular composition of the gelatin in any of the exemplary embodiments may vary depending upon the intended use of the encapsulation composition.

In another embodiment, the gelatin composition includes gelatin in an aqueous solution containing from about 10% to about 15% gelatin by weight, from about 5% to about 15% calcium carbonate by weight, and from about 1% to about 5% gelatin hydrolysate by weight. The particular composition of the gelatin in any of the exemplary embodiments may vary depending upon the intended use of the encapsulation composition, as described in Section III above.

In yet another embodiment, the gelatin composition includes gelatin in an aqueous solution containing from about 36% to about 42% gelatin by weight, from about 17% to about 22% plasticizer by weight, from about 26% to about 31% water by weight, and from about 10% to about 15% calcium carbonate by weight. The particular composition of the gelatin in any of the exemplary embodiments may vary depending upon the intended use of the encapsulation composition.

In a further embodiment, the gelatin composition includes gelatin in an aqueous solution containing from about 32% to about 40% gelatin by weight, from about 17% to about 22% plasticizer by weight, from about 26% to about 31% water by weight, from about 10% to about 15% calcium carbonate by weight, and from about 2% to about 6% gelatin hydrolysate by weight. The particular composition of the gelatin in any of the exemplary embodiments may vary depending upon the intended use of the encapsulation composition.

Any of the gelatin compositions described above may be used in the production of a variety of therapeutic compositions that include the gelatin capsule composition. Non-limiting examples of therapeutic composition embodiments include rapid-release coated tablets, soft-gel capsules, hard capsule shells, and chewable therapeutic compositions.

In one embodiment, the currently claimed gelatin compositions include a rapid-release tablet composition wherein the outer surface coating is applied as a thin coat over the outer surface of an active ingredient in solid tablet form, which forms the core. The outer surface coating may be applied to the active ingredient using any technique known in the art, including, but not limited to, pan coating, drum coating, film coating, spray coating, and dip coating.

In a further embodiment, the currently claimed gelatin compositions include a rapid-release soft-gel capsule wherein the outer surface coating forms a continuous membrane that encloses the active ingredient, which may be in a liquid or powder form. The gelatin composition may be formed into a gel-cap using any technique known in the art, including, but not limited to, forming and filling individual gel-caps in a mold, formation of the gel-caps using a rotary die and filling using blow molding, or Accogel-type encapsulation techniques. In these embodiments, the gelatin composition may further include a plasticizer including but not limited to glycerin, mixtures of sorbitol derivatives or mixtures thereof.

In still another embodiment, the currently claimed gelatin compositions include a hard capsule composition wherein the outer surface coating forms a rigid shell that encloses the active ingredient core, which may be in a liquid, granular, or powder form. The hard capsule composition may be in a form including, but not limited to, a continuous shell formed around the active ingredient, or two telescopically-joined half-shells in which each half-shell is formed separately, and the active ingredient is inserted prior to joining the half-shells. The gelatin composition may be formed into a hard capsule using any technique known in the art, including, but not limited to, dip-coating metal rod ends, or injection molding.

The gelatin compositions of the currently claimed embodiments may further include a polymer as incorporated into the outer surface coating, core, or both. As used herein, the term “polymer” is understood by those skilled in the art to generally encompass any molecule composed of repeating monomer structural units, including but not limited to homopolymers, copolymers, heteropolymers, branched polymers, branched copolymers, star copolymers, brush copolymers, comb copolymers, graft copolymers, and block copolymers. The polymer may include any suitable polymer known in the art including, but not limited to, synthetic polyvinyl polymers, synthetic polyethylene polymers, synthetic acrylic polymers, biopolymers, modified biopolymers, and combinations thereof. Suitable synthetic polyvinyl polymers include but are not limited to polyvinylchloride, polyvinylacetate and copolymers thereof, polyvinylalcohol, and polyvinylpyrrolidone. Synthetic polyethylene polymers may include but are not limited to polyethylene and polystyrene. Synthetic acrylic polymers may include but are not limited to methylmethacrylate or copolymers of acrylic monomers. Non-limiting examples of biopolymers and modified biopolymers include ethylcellulose, cellulose acetate phthalate, cellulose acetate, hydroxypropyl cellulose, hydroxypropylmethyl cellulose, methylcellulose, microcrystalline cellulose, carboxymethyl cellulose, sodiumcarboxymethyl cellulose, shellac, gelatin, ethylcellulose, cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate and cellulose acetate butyrate. Other suitable polymers are described in U.S. Pat. Nos. 3,845,770; 3,916,899; 4,008,719; 4,036,228 and 4,612,008 (which are incorporated herein in their entirety by reference).

In one embodiment, for purposes of fabricating various gelatin composition embodiments using an aqueous solution, a water-soluble polymer may be suitable for various embodiments, as many formulations, including, but not limited to, tablet coatings, soft gelatin capsules, and hard capsules are generally formed from a liquid polymer solution. The water-soluble polymers may be soluble in aqueous solution at a pH ranging from about 6 to about 8. Non-limiting examples of water-soluble polymers include carboxymethylcellulose, cross-linked polyvinylpyrrolidone, hydroxylpropylcellulose, hydroxypropyl methylcellulose, microcrystalline cellulose, shellac, and gelatin. In particular, the gelatin capsule composition may include gelatin and carboxymethylcellulose.

In an alternative aspect of the currently claimed embodiments, the gelatin composition comprises a core, a subcoating, and an outer surface coating. In this embodiment, the subcoating is located distally to the core, but proximal to the outer surface coating exposed to the external environment. The use of subcoatings is well known in the art and disclosed in, for example, U.S. Pat. No. 5,234,099, which is incorporated by reference herein. Any composition suitable for film-coating a tablet may be used as a subcoating according to the present invention. Examples of suitable subcoatings are disclosed in U.S. Pat. Nos. 4,683,256, 4,543,370, 4,643,894, 4,828,841, 4,725,441, 4,802,924, 5,630,871, and 6,274,162, which are all incorporated by reference herein. Suitable compositions for use as subcoatings include those manufactured by Colorcon, a division of Berwind Pharmaceutical Services, Inc., 415 Moyer Blvd., West Point, Pa. 19486 under the tradename “OPADRY®” (a dry concentrate comprising film forming polymer and optionally plasticizer, colorant, and other useful excipients).

Additional suitable subcoatings include one or more of the following ingredients: cellulose ethers such as hydroxypropylmethylcellulose, hydroxypropylcellulose, and hydroxyethylcellulose; polycarbohydrates such as xanthan gum, starch, and maltodextrin; plasticizers including for example, glycerin, polyethylene glycol, propylene glycol, dibutyl sebecate, triethyl citrate, vegetable oils such as castor oil, surfactants such as Polysorbate-80, sodium lauryl sulfate and dioctyl-sodium sulfosuccinate; polycarbohydrates, pigments, and opacifiers. The subcoating can be applied as a clear, transparent coating such that the core can be seen.

The subcoating may be applied to the entire exterior surface of the core or may be applied to a portion less than the entire surface of the core, depending on the desired properties of the gelatin composition. In another embodiment, the subcoating may include one or more passageways for the controlled release of the contents of the core to the external environment after the outer surface coating has dissolved and/or separated from the subcoating. As used herein the term “passageway” includes an aperture, orifice, bore, hole, weakened area or an erodible element such as a gelatin plug that erodes to form a channel for the release of the contents of the core from the dosage form. Passageways used in accordance with the subject invention are well known and are described in U.S. Pat. Nos. 3,845,770; 3,916,899; 4,034,758; 4,077,407; 4,783,337 and 5,071,607. In one embodiment the one or more passageways include laser drilled holes in the subcoating.

In yet another aspect of the currently claimed embodiments, the gelatin composition comprises an enteric coating, an outer surface coating, and a core, wherein the enteric coating is applied to the distal surface of the outer surface coating and the outer surface coating is applied to the distal surface of the core. As used herein, the term “enteric coating” represents coatings that are insoluble in a low pH environment such as the stomach, where the fluid has a pH value normally less than about 3.5, but dissolve rapidly or swell sufficiently to disintegrate in a high pH environment, such as intestinal fluid, which has a pH value normally greater than about 5.0. The polymers typically used for enteric coatings are well known within the art, as described in U.S. Pat. Nos. 3,391,135, 3,629,237, 4,017,647, 4,138,013, 4,960,814, and 5,356,634, all of which are incorporated herein by reference in their entirety. Non-limiting examples of enteric polymers include, but are not limited to cellulose acetate phthalate (C-A-P), cellulose acetate trimellitate (C-A-T), hydroxypropylmethylcellulose phthalate (HPMCP), copolymer of methacrylic acid and ethyl acrylate, hydroxypropylmethylcellulose acetate succinate (HPMCAS), and polyvinyl acetate phthalate (PVAP).

The compounds and processes of the invention will be better understood by reference to the following examples, which are intended as an illustration of and not a limitation upon the scope of the invention. Each example illustrates at least one method of preparing various intermediate compounds and further illustrates each intermediate utilized in the overall process. These are certain preferred embodiments, which are not intended to limit the present invention's scope. On the contrary, the present invention covers all alternatives, modifications, and equivalents as can be included within the scope of the claims and routine experimentation.

### EXAMPLES

The following examples illustrate various aspects of the invention.

### Example 1

### Effect of Production Method on Dissolution Properties of Gelatin Compositions

To assess the effect of the addition of acid compounds used in the production of gelatin encapsulant compositions on the dissolution properties of the compositions, the following experiment was conducted.

600 grams of pigskin gelatin was dissolved in 3400 g of deionized water, and the mixture was filtered through a mixed bed of ion-exchange resin (pH>9). The gelatin mixture was then divided into five parts and adjusted to a pH of 5.5±0.1 using the following acids: sulfuric acid, tartaric acid, citric acid, acetic acid, and carbon dioxide. The carbon dioxide was supplied either by the sublimation of dry ice or using sodium bicarbonate. Each of the five mixtures was chilled overnight and then dried in dehydrators. In the case of treatment with carbon dioxide, a pH of 5.5 was not attained but the pH was significantly reduced from pH 9.

The dried samples were ground and prepared as a coating. A 30% gelatin solution of each sample was prepared by weighing 50 grams of the dried gelatin into a 250 ml beaker, adding 116.7 g of de-ionized water, and stirring to mix. After mixing, the beaker was covered with a watch glass and allowed to swell for about 1 hour at room temperature. The mixture was then melted at 60° C. for about 4 hours, stirring after about 1 hour stir to mix. A series of glass plates were preheated to a temperature of about 60° C. and loaded into an automated coating device. After removing any skin or bubbles from the surface of the melted gelatin mixture, the mixture was loaded into the automated coating device and coated on to the series of preheated glass plates. The films were stored overnight (approximately 17 hours) in a temperature and humidity controlled room at 45%±5% RH and 70°±5° F.

For each of the five compositions, a sample of the coating weighing approximately 0.075 g was placed into each of six reaction vessels filled with 900 g of KH₂PO₄ solution having a pH of 3.0 and heated to 37° C. The absorptivity of the reaction vessel contents at a wavelength of 218 nm was used to measure the dissolution of the coating samples over a period of about 15 minutes.

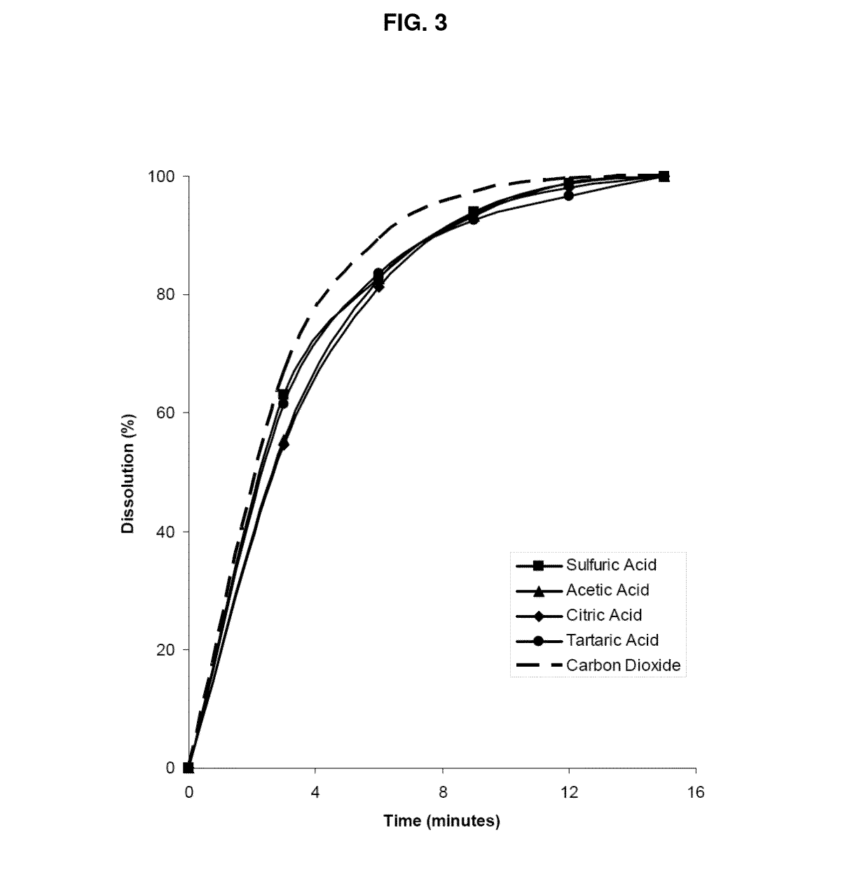

The results of the dissolution measurements are summarized in FIG. 3. The dissolution curve for the coating composition that incorporated carbon dioxide in the form of sodium bicarbonate or dry ice did not significantly affect the dissolution properties of this composition relative to any of the other coating compositions. However, in this experiment, sodium bicarbonate was not added to the coating composition in an amount sufficient to induce the formation of CO₂ gas bubbles during the dissolution of the composition.

The results of this experiment demonstrated that the dissolution rates of the coating compositions tested in this experiment were sensitive to the composition of the coating. In particular, the dissolution properties of the coating composition that incorporated carbon dioxide produced using dry ice or sodium bicarbonate in the amounts specified by this experiment were not significantly different than any of the other coating compositions tested.

### Example 2

### Effect of Sodium Bicarbonate Added During Production of Gelatin Encapsulation Compositions on Dissolution Properties

To assess the effect of adding sodium bicarbonate during the production of a gelatin encapsulation composition on the dissolution properties of the resulting gelatin coating, the following experiment was conducted.

600 grams of gelatin was dissolved in 3400 g of deionized water, and the mixture was filtered through a mixed bed of ion-exchange resin (pH>9). The gelatin mixture was then adjusted to a pH of 5.5±0.1 using sulfuric acid. The gelatin mixture was then divided into two halves, and 2% sodium bicarbonate by weight was added to one half of the gelatin mixture. Each of the two mixtures was chilled overnight and then dried in dehydrators. Coating samples were formed from the two mixtures using the methods described in Example 1.

A buffer solution having a pH of 1.0 was formed by adding 200 ml of 2M HCl and 29.8 g of KCl to 1800 ml of deionized water. Coating samples from the two gelatin mixtures were added to two sets of six reaction vessels containing 900 g of the pH=1 buffer solution and the dissolution of the coating samples was measured using the method previously described in Example 1.

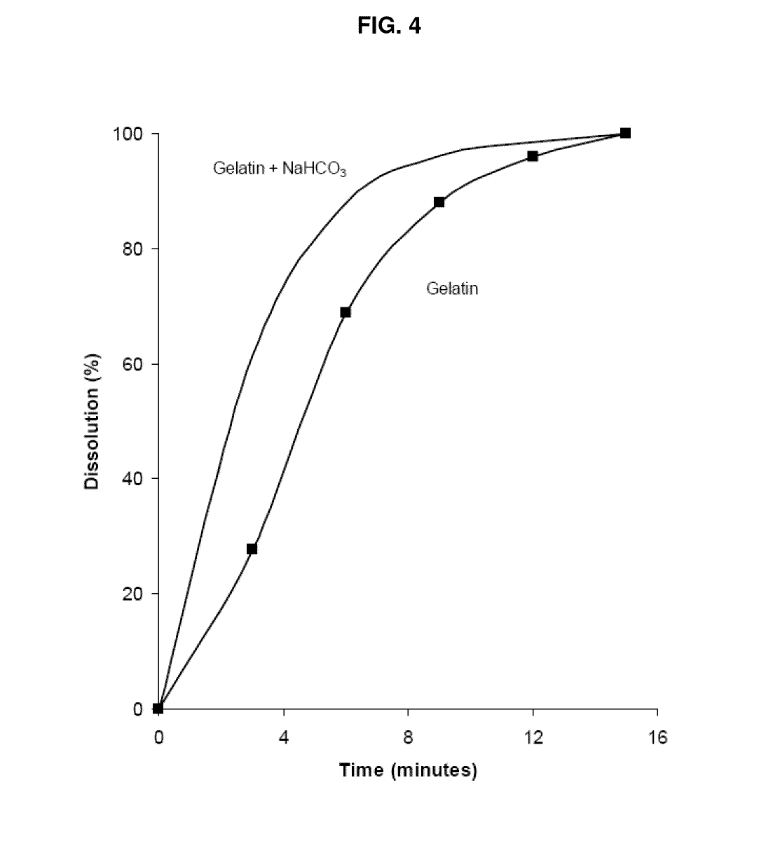

The measured dissolutions of the coating samples are summarized in FIG. 4. The addition of sodium bicarbonate to the gelatin mixture during the production of the gelatin encapsulant composition significantly increased the rate of dissolution of the resulting gelatin coating relative to the same gelatin coating produced without any added sodium bicarbonate. In particular, the addition of sodium bicarbonate significantly increased the rate of tearing apart of the film during the procedure.

The results of this experiment demonstrated that the addition of sodium bicarbonate during the production of a gelatin coating composition significantly increased the rate of dissolution of the resulting gelatin coating compared to the same gelatin coating composition that lacked sodium carbonate, largely due to the increased rate of tearing apart of the film due to carbon dioxide bubbles formed from the sodium carbonate during the procedure.

### Example 3

### Effect of Carbonate Compounds on pH of Gelatin Mixtures

To assess the sensitivity of the pH of a deionized gelatin suspension to the addition of various carbonate compounds, the following experiment was conducted. A deionized gelatin suspension was formed using the methods described in Example 1 and sulfuric acid was added to the gelatin suspension to adjust the pH of the suspension to about 4.7. The gelatin suspension was divided into three equal parts. To the first part of the gelatin suspension, 10% calcium carbonate by weight was added. To the second and third parts of the gelatin suspension, sodium bicarbonate in the amount of 5% and 7.5% by weight was added, respectively. The pH of each gelatin suspension was measured before and after the addition of the carbonate, and is summarized in Table 1 below:

TABLE 1
pH of Gelatin Suspensions Before and After Addition of Carbonates

| | pH of Gelatin Suspension | |
|---|---|---|
| Carbonate Added to | Before Addition | After Addition of |
| Suspension | of Carbonate | Carbonate |
| 10% CaCO₃ (wt %) | 4.7 | 7 |
| 5% NaHCO₃ (wt %) | 4.7 | 7.59 |
| 7.5% NaHCO₃ (wt %) | 4.7 | 7.75 |

The addition of the carbonates to the gelatin mixtures generally increased the pH of the gelatin suspension as expected due to the basic properties of carbonates in solution. However, the addition of calcium carbonate increased the pH of the gelatin suspension significantly less than the addition of sodium bicarbonate, despite adding a higher amount of calcium carbonate. This was most likely due to the lower solubility of the calcium carbonate at the original pH of the gelatin suspension (pH=4.7) compared to sodium bicarbonate. Because less calcium carbonate underwent dissociation in the gelatin mixture, less neutralization of the gelatin suspension occurred, resulting in the maintenance of a lower pH in the gelatin suspension after the addition of the calcium carbonate.

The results of this experiment demonstrated that the pH of the gelatin suspensions tested were sensitive to the amount and composition of the carbonate added to the suspension.

### Example 4

### Effect of Extended Storage at Elevated Temperature and Humidity on the Dissolution Properties of Gelatin Encapsulation Compositions

To assess the sensitivity of the dissolution properties of several gelatin encapsulation compositions to extended storage times at elevated temperature and humidity, the following experiment was conducted. A deionized bone gelatin suspension was formed and used as an encapsulation composition using methods similar to those described in Example 1. The gelatin suspension was divided into three equal parts and used to form three different encapsulation compositions. The first encapsulation composition included the bone gelatin without further modification (CONTROL). The second encapsulation composition included the bone gelatin as well as 15% CaCO₃ by weight (FD—fast-dissolving). The third encapsulation composition included the bone gelatin, 15% CaCO₃ by weight, and 10% by weight of hydrolyzed bone gelatin having a molecular weight of about 500 Daltons (FD+SH). The dissolution of each of the three encapsulation compositions in deionized water and in a pH=1 buffer solution were measured using the method described in Example 1. The dissolutions of the encapsulation compositions were measured immediately after the compositions were produced, as well as after storage at 50° C. and 80% relative humidity for periods of two, five and eleven weeks.

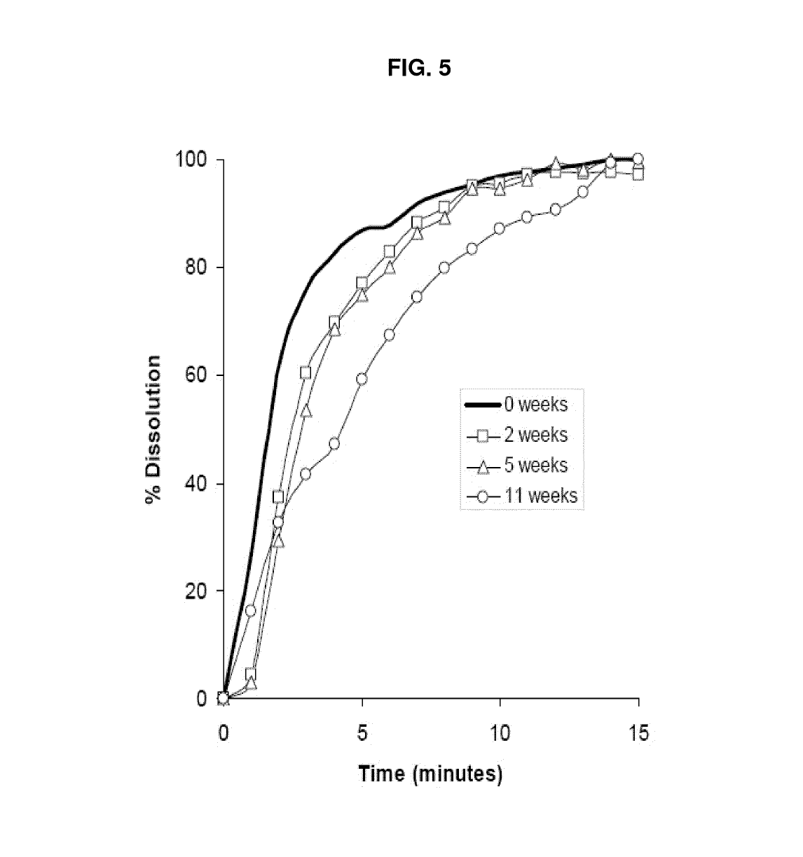

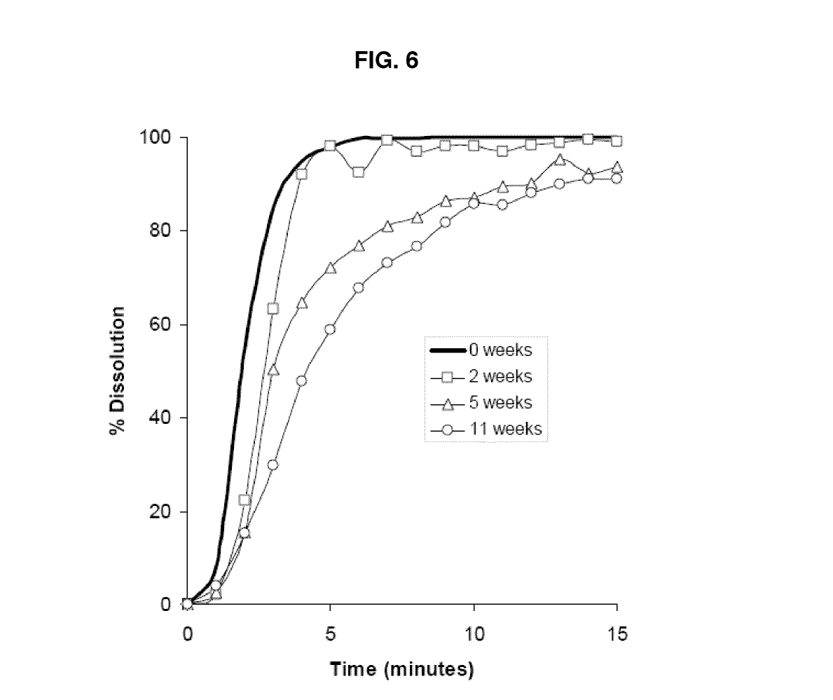

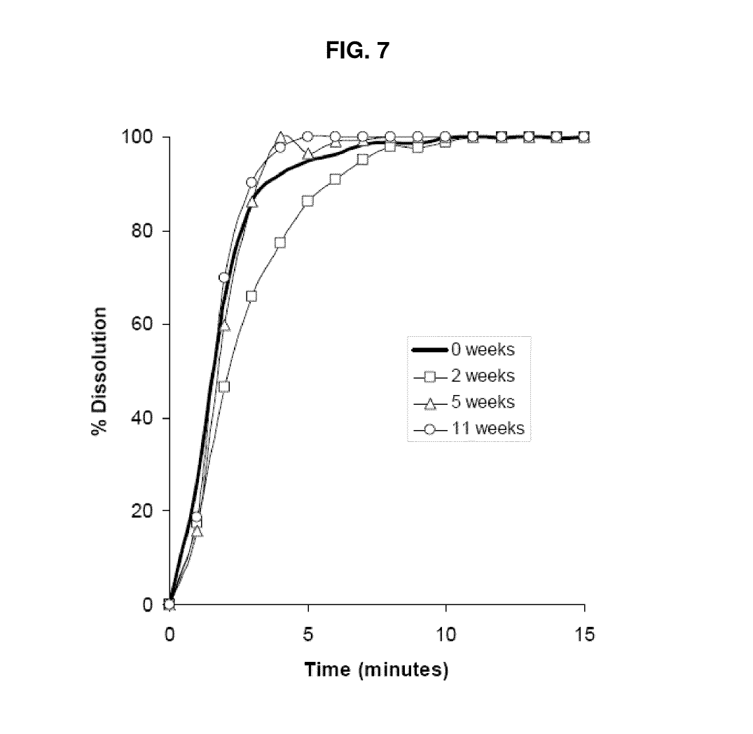

The dissolution results for the CONTROL, FD, and FD+SH encapsulation compositions in pH=1 buffer solution are summarized in FIGS. 5, 6, and 7 respectively. FIGS. 5 and 6 both show a marked reduction in the dissolution rate at longer periods of storage at 50° C. and 80% relative humidity. Although FIG. 6 exhibits a similar trend of reduction of dissolution rate for the FD+SH encapsulation composition after two weeks of storage, the dissolution rate returns to levels similar to initial dissolution rates after five and eleven weeks of storage. FIG. 8 is a comparison of the dissolution results of the three encapsulation compositions in pH=1 buffer solution after eleven weeks of storage. The dissolution rate of the FD+SH composition is maintained at a significantly higher level than either the CONTROL or FD compositions.

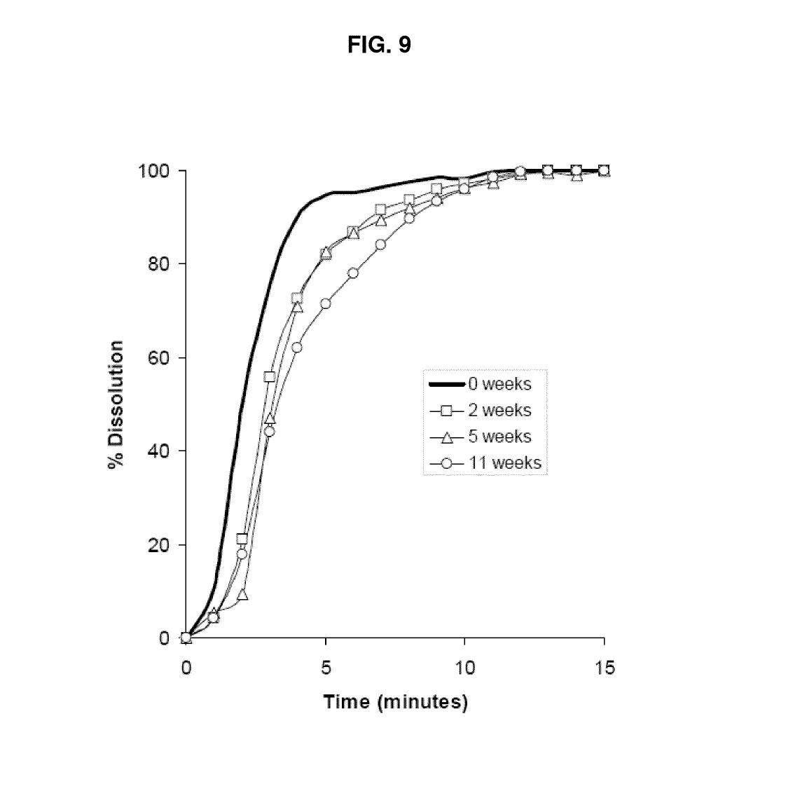

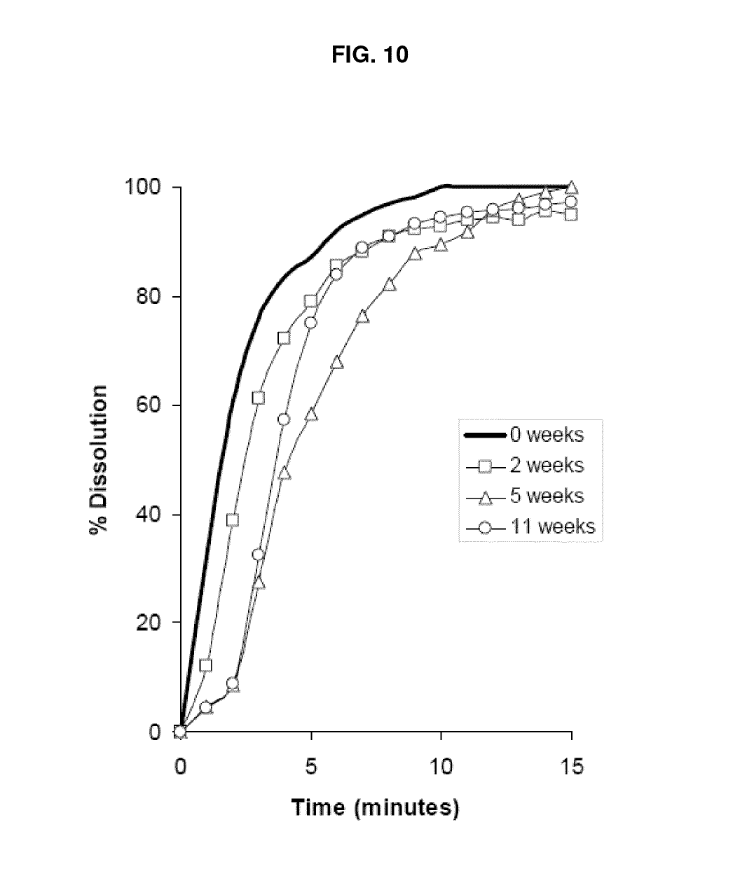

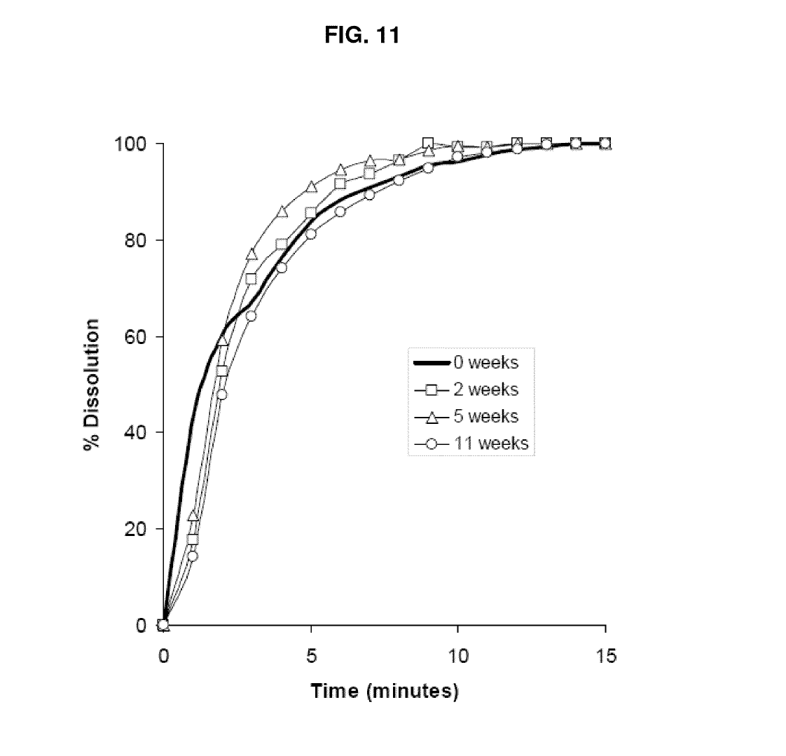

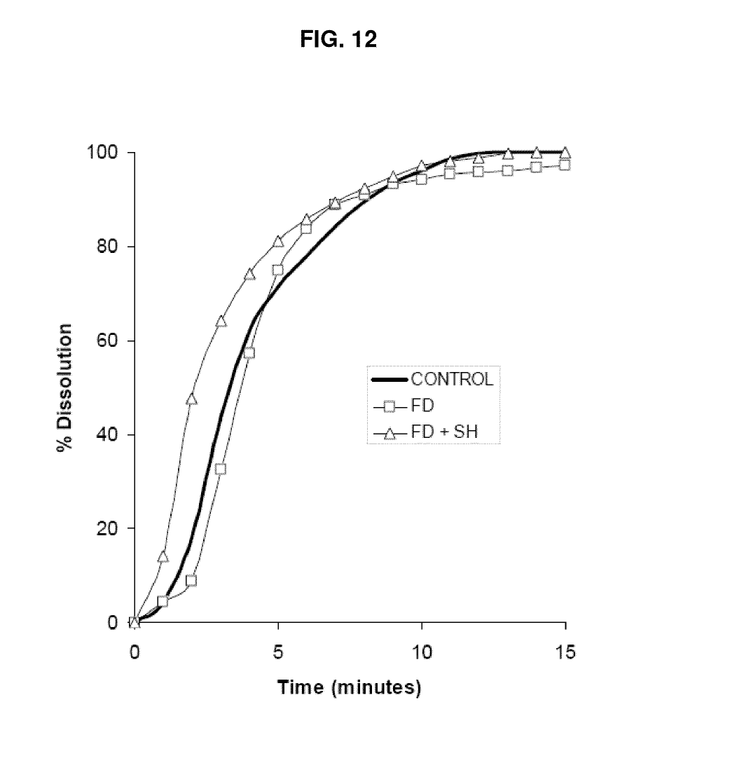

The dissolution results for the CONTROL, FD, and FD+SH encapsulation compositions in deionized water are summarized in FIGS. 9, 10, and 11 respectively. FIGS. 9 and 10 both show degradations of dissolution rate for the CONTROL and FD compositions after extended periods of storage in a manner similar to the degradations shown in FIGS. 5 and 6. As shown in FIG. 11, the dissolution rate of the FD+SH composition in deionized water was essentially unaffected by periods of extended storage at 50° C. and 80% relative humidity. FIG. 12 is a comparison of the dissolution results for the CONTROL, FD, and FD+SH compositions in deionized water after 11 weeks of storage, clearly showing that the FD+SH composition maintains a significantly higher dissolution rate even after eleven weeks of storage.

The reduction in dissolution rate in the CONTROL and FD encapsulation compositions after storage at elevated temperature and humidity conditions is likely due to the formation of cross-bridges within the gelatin in the encapsulation compositions. The addition of low molecular weight hydrolysates having molecular weights from about 100 to about 2000 Daltons to the encapsulation composition may interfere with the formation of cross-bridges, thereby maintaining the dissolution characteristics of the gelatin encapsulation compositions at initial levels, even after extended periods of storage.

The results of this experiment demonstrated that the dissolution properties of gelatin encapsulation compositions may degrade after storage at elevated temperatures and humidity. This degradation is sensitive to the particular composition, and the degradation may be virtually eliminated by the addition of hydrolysates having molecular weights from about 100 to about 2000 Daltons to the gelatin encapsulation compositions.

### Example 5

### Effect of Extended Storage at Elevated Temperature and Humidity on Dissolution Properties

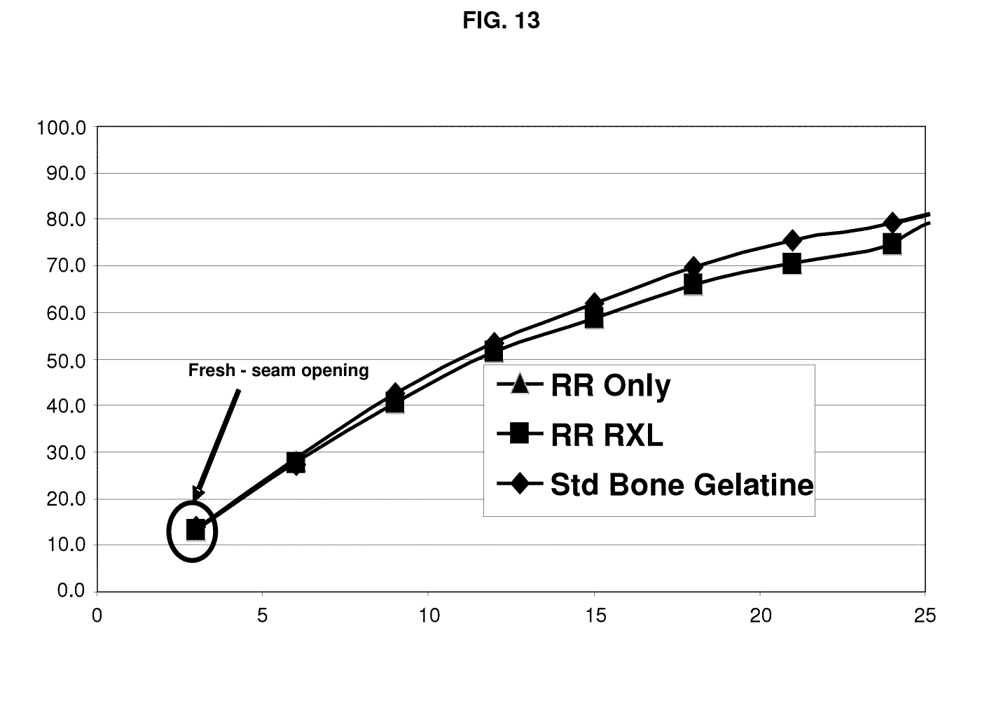

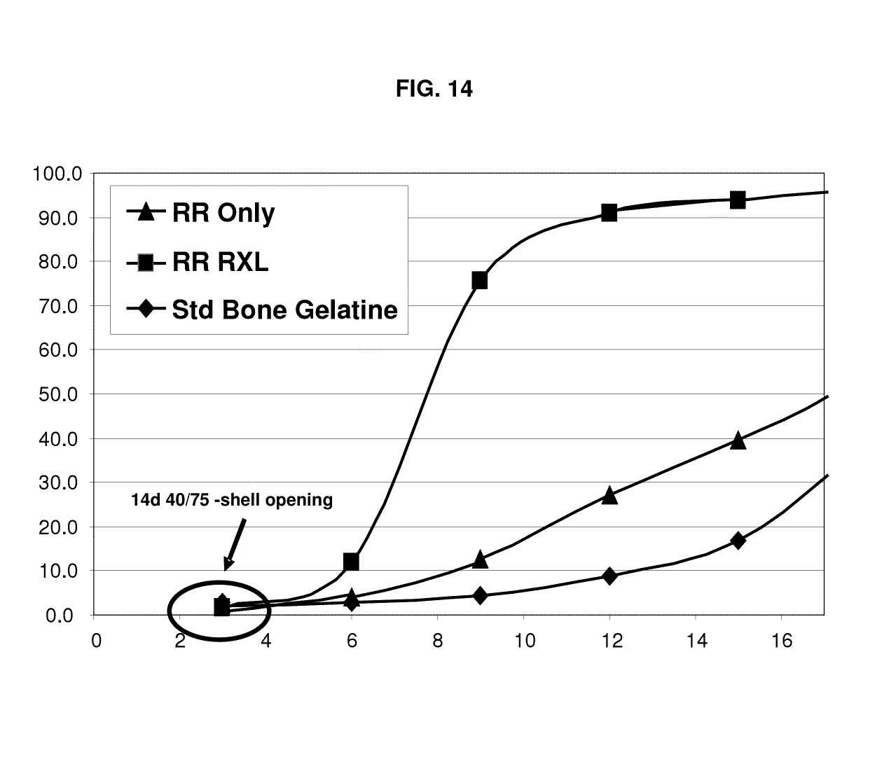

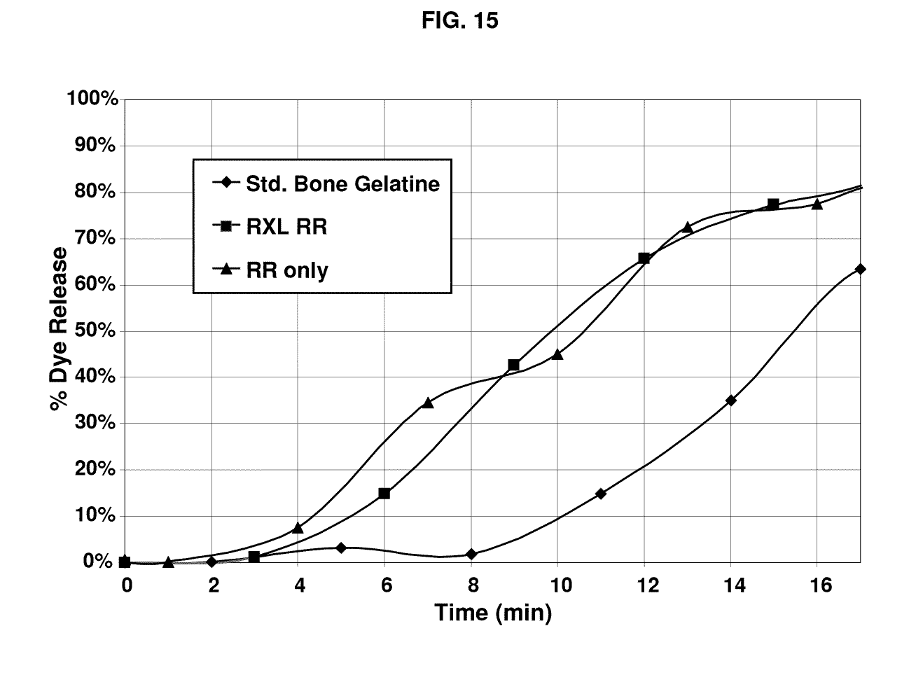

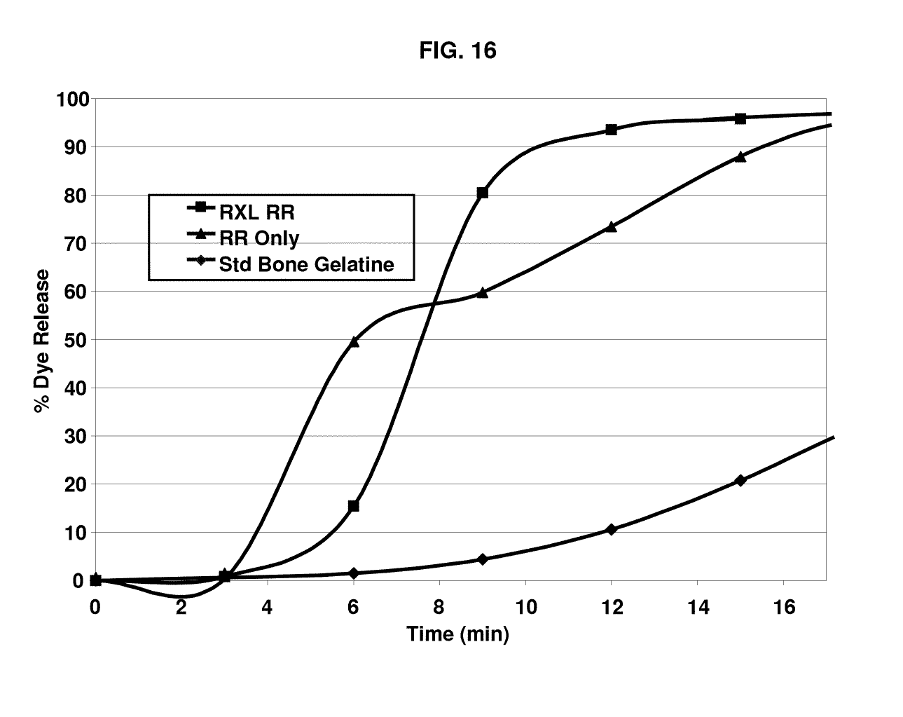

To further assess the dissolution properties of the various gelatin formulations discussed herein after exposure to temperature and humidity conditions, the following soft capsule experiment was conducted. Soft capsule encapsulation compositions were prepared having viscosities at 60° C. of about 10,000 mPas. The first encapsulation composition included the bone gelatin without any further modifications at 43.00 weight percent, sorbitol at 10.75%, glycerol at 10.75%, and water at 35.5% (“Std Bone Gelatin”). The second encapsulation composition included the bone gelatin at 40.8%, calcium carbonate (CaCO₃) at 7.2%, sorbitol at 10.2%, glycerol at 10.2%, and water at 31.6% (“RR only”). The third encapsulation composition included the bone gelatin at 38.35%, hydrolyzed bone gelatin having a molecular weight of about 500 Daltons at 2.45%, calcium carbonate (CaCO₃) at 7.2%, sorbitol at 10.2%, glycerol at 10.2%, and water at 31.6% (“RR RXL”). The three encapsulation compositions were utilized to make soft capsules on a Modified Chan Sung soft capsule machine Type M3 having dual cavity dye rolls to make 7.5 oval capsules and operating at 2.5 rpm with 30 minute tumble drying followed by one week room drying at about 25° C. and about 35% RH. The capsules were filled with a liquid formulation comprising polyethylene-glycol at 96.51%, glycerol at 2.99%, and brilliant blue dye at 0.50% (the fill). The three formulations were tested to determine the dissolution profile over time for three distinct dissolution mediums. In each case, the dissolution media were monitored spectrophotometrically to observe the appearance of brilliant blue dye in the media. The first test assessed the dissolution profile for fresh capsules of the three formulations in simulated gastric fluid (at a pH of approximately 1.3, and in the absence of any enzymes). The results of the first test are illustrated in FIG. 13. The second test assessed the dissolution profile of the three formulations in water (approximately neutral pH levels) after the formulations were stored at 40° C. and 75% relative humidity for a period of two weeks. The results of this second test are illustrated in FIG. 14. The third test assessed the dissolution profile of the three formulations in simulated gastric fluid (at a pH of approximately 1.3, and in the absence of any enzymes), after the formulations were stored at 40° C. and 75% relative humidity for a period of two weeks and four weeks. The results of this test are illustrated in FIGS. 15 and 16 for the two week storage and four week storage periods, respectively.

In reference to the results of the first test, illustrated in FIG. 13, the chart shows that little difference in dissolution profile exists for the Std Bone Gelatin, RR only, and RR RXL formulations. This result was expected as the capsules readily opened along the seam, releasing the dye, when they were fresh, immediately after they are produced.

The results of the second test, illustrated in FIG. 14, depict a difference in dissolution profiles for the three formulations after storage for two weeks. Specifically, FIG. 14 shows that the RR RXL exhibited a substantially faster and more complete dissolution profile compared to the RR only and Std Bone Gelatin formulations. The results in FIG. 14 illustrate the impact of incorporating the hydrolyzed gelatin component, which improves dissolution even though the formulations were tested in a neutral water solution. The RR Only formulation showed some improvement over the dissolution profile of the Std Bone Gelatin formulation; however, the results were not as robust as the RR RXL formulation. This is not unexpected as the dissolution medium was a neutral water solution, so the calcium carbonate was not exposed to pH levels that would cause it to effervesce and further advance dissolution of the formulation. It is important to note that the dissolution profile for the Std Bone Gelatin formulation showed a slower rate of dissolution and decreased overall dissolution, compared to the results of the first test. This is evidence that, upon storage at increased temperature (40° C.) and humidity (75% relative humidity), the dissolution properties of standard gelatin formulations is adversely affected.

The results of the third test, illustrated in FIGS. 15 and 16, depict an improved dissolution profile for the RR RXL and RR Only formulations as compared to the Std Bone Gelatin formulation. Specifically, the RR RXL and RR Only formulations exhibited a more rapid dissolution rate, as well as more complete dissolution profile, approaching 100% dissolution by the end of the time periods tested. These results illustrate the effect of incorporating calcium carbonate (in the RR RXL and RR Only formulations) and hydrolyzed gelatin (in the RR RXL formulation) on dissolution rates. It is also important to note that the dissolution profile for the Std Bone Gelatin formulation showed a slower rate of dissolution and decreased overall dissolution, compared to the results of the first test, especially in FIG. 16, illustrating dissolution after four weeks of storage. This is evidence that, upon storage at increased temperature (40° C.) and humidity (75% relative humidity), the dissolution properties of standard gelatin formulations is adversely affected.

### Example 6

### Production of a Gelatin Capsule with Distinct Regions Comprising a Water-Insoluble Rapid Release Agent

A solution of gelatin (termed Sample A) containing approximately 7.5% of a gelatin hydrolysate having average molecular weight of less than 1,000 Daltons was prepared and the pH was adjusted to 6.8 using 50% sodium hydroxide. Specifically, a gelatin solution comprising 1546.4 grams of gelatin, including 115.9 grams gelatin hydrolysate was developed. The solution was sterilized and a portion comprising 895.4 grams of the solution was dried, forming Sample A. The remaining portion of the gelatin solution (651 grams) was utilized to prepare Sample B. Specifically, to the remaining 651 grams of gelatin solution not incorporated into Sample A, 434 grams of calcium carbonate (Vicality™ Medium, mean particle size 1.9 microns, USP Grade) was added to attain a ratio of (dry) gelatin to (dry) calcium carbonate of approximately 3 to 2. The slurry was dried and the resulting combination was deemed Sample B. It is noted that the solutions were sterilized using a standard steam injection process whereby live, filtered steam is injected into the product, and the solution is maintained at a temperature of about 240° C. for a period of about 8 seconds, thereby killing any microorganisms present in the solution.

Subsequent to development, Sample A and Sample B were tested to determine the physical characteristics of each compound. Specifically, bloom strength, viscosity, moisture content, ash content, and calcium concentration were tested. The results are illustrated in Table 2 below.

TABLE 2
Physical Characteristics of Samples A and B.

| | | Viscosity | Moisture | | Calcium |
|---|---|---|---|---|---|
| | Bloom | (mp) | (%) | Ash (%) | (ppm) |
| Sample A | 125 | 24.8 | 12.8 | 0.81 | 48.2 |
| Sample B | 63 | 12.4 | 8.4 | 21.28 | 159740 |

After testing of Sample A and Sample B was performed to determine the physical characteristics of both gelatin solutions, each sample was incorporated into a gel mass solution for the development of gelatin coatings. Specifically, Sample A and Sample B were mixed with glycerin and water in the proportions found in Table 3, and subsequently de-bubbled to remove any air particles from the samples.

TABLE 3
Gel Mass Solutions

| | Weight of sample | | |
|---|---|---|---|
| | (g) | Glycerin (g) | Water (g) |
| Sample A | 895.4 | 412.5 | 754.6 |
| Sample B | 1085 | 300 | 548.8 |

After the gel mass solutions of Sample A and Sample B were developed, gelatin coatings having alternating stripes of gel mass solutions of Sample A and Sample B were produced by pumping each solution simultaneously with each other at equal rates through a Kenics static mixer having 4 orthogonal helical mixing units and a pipe diameter of ⅜ inches. The resulting interleaved mixture was coated on polycarbonate plates using a coating bar having a coating gap of 500 μm. The resulting striped coatings were dried and stored in a constant humidity chamber at 22° C. and 50% R.H.

The gelatin coatings were subsequently tested to determine dissolution rates. Specifically, the striped coatings were peeled off the polycarbonate substrate and tested for dissolution properties. A sample of the striped coating (usually approximately 1 cm×1 cm) was cut from the coating and the sample was floated on the surface of the dissolution medium to simulate one-sided exposure of the coating to the dissolution medium. The dissolution medium was prepared to simulate stomach acid (without enzymes) according to the following procedure. Two grams of sodium chloride and 7.0 ml of concentrated HCl were dissolved in sufficient water to make 1000 ml. The pH of the dissolution medium was approximately 1.2. The dissolution medium was warmed to 37° C. for the dissolution testing.

Each time the striped coatings were tested for dissolution rates, the gelatin coating preferentially disintegrated along the “stripes” constituting Sample B (incorporating the calcium carbonate) and the portions of the gelatin coating constituting Sample A (comprising only gelatin and gelatin hydrolysate, without calcium carbonate) dissolved at a slower rate. It was observed that once the stripes disintegrated, the un-dissolved portions of the coating constituting Sample A separated from one another, causing the coating to rapidly come apart in fewer than 2 minutes. This separation of the un-dissolved portions of Sample A is important as it simulates the ability of the striped coating to rapidly tear the gelatin coating, thereby allowing the internal contents of the capsule or coating (if present) to be rapidly released. The striped gelatin coatings were tested at multiple time periods after formation to determine if prolonged storage periods would affect dissolution rates. The dissolution times are found in Table 4 below.

TABLE 4
Dissolution Times for Striped Gelatin Coatings

| Striped capsule storage time | Disintegration time (min) |
|---|---|
| 1 day | <2 minutes |
| 1 week | <2 minutes |
| 2 weeks | <2 minutes |
| 3 weeks | <2 minutes |
| 6 weeks | <2 minutes |
| 11 weeks | <2 minutes |

Thus, as is evident from Table 4, prolonged storage of the striped gelatin coatings did not have an adverse impact on the dissolution characteristics of the coatings. At all time points tested, including 1 day, 1 week, 2 weeks, 3 weeks, 6 weeks, and 11 weeks after development, the striped gelatin coatings maintained rapid dissolution times of less than 2 minutes, suggesting that storage for prolonged periods of time does not affect the dissolution characteristics

As a comparison, gelatin coatings comprising only Sample A gel mass solution were prepared and tested for dissolution after storage for 2 weeks and 4 weeks at 22° C. and 50% R.H. The pure Sample A coatings were immersed in the dissolution media (two-sided contact with the dissolution media) and approximately 66% of the gelatin had dissolved in 3 minutes and no disintegration or separation of the film was observed until the coating had fully dissolved in about 9 minutes. Accordingly, the striped gelatin coatings comprising calcium carbonate “stripes” demonstrated more rapid dissolution and separation of the gelatin coating as compared to the dissolution and separation of the gelatin coating comprising only gelatin and gelatin hydrolysate.

### Example 7

### Effect of Adding Carbonate Salts to Bone Gelatine on pH Values of Composition

In order to assess the affect on pH of adding water-soluble rapid release agents to gelatin formulations, the following experiment was performed. 600 grams of limed bone gelatine were dissolved in 3400 ml of water. The mixture was developed at a temperature of 60° C., and the pH for the formulation prior to addition of any water-soluble carbonate salts was measured to be 5.468 (“the initial pH”). Four equal portions of 1000 grams of the gelatine and water solutions were formed, labeled A, B, C, and D. For formulations A and B, a total of 6 grams and 12 grams of sodium bicarbonate were added to each formulation, respectively, and, after the bubbling subsided, the pH of formulation A and B at 60° C. was recorded. In addition, for formulations C and D, a total of 6 grams and 12 grams of sodium carbonate were added to each formulation, respectively, and, after the bubbling subsided, the pH of formulation C and D at 60° C. was recorded. The pH values measured immediately after formulation and bubbling subsided was noted as “pH Immediately After Formulation.” Furthermore, each of formulations A, B, C, and D was stored overnight at room temperature, and pH was again measured on day 2 at 60° C. The pH values measured on day 2 were noted as “pH After Storage Overnight.”

TABLE 2
pH Values for Sodium Carbonate and Sodium Bicarbonate Added
to Gelatin Formulations

| | pH Immediately | | |
|---|---|---|---|
| | After | pH After Storage | |
| Formulation | Formulation | Overnight | Average pH |
| A | 6.831 | 6.875 | 6.853 |
| B | 7.156 | 7.213 | 7.1845 |
| C | 9.246 | 9.229 | 9.2375 |
| D | 9.800 | 9.852 | 9.826 |

As illustrated in Table 2, the addition of water-soluble carbonate salts to the gelatin formulations resulted in a substantial rise in pH for each of the formulations when compared to the initial pH value (prior to introduction of the carbonate salts) of 5.468. Specifically, Formulations A and B, incorporating 6 grams and 12 grams of sodium bicarbonate, respectively, and formulations C and D, incorporating 6 grams and 12 grams of sodium carbonate, respectively, all demonstrated a significant increase in the pH level after the incorporation of carbonate salt. The data in Table 2 also demonstrates that the formulations incorporating greater amounts of the carbonate salt (formulations B and D, incorporating 12 grams of sodium bicarbonate and sodium carbonate, respectively) demonstrated a greater increase in pH.

### Example 8

### Production of Rapid-Release Encapsulation Compositions Comprising a Water-Soluble Rapid Release Agent

A solution of gelatin (termed Sample A) containing approximately 7.5% of a gelatin hydrolysate having average molecular weight of less than 1,000 Daltons was prepared and the pH was adjusted to 6.8 using 50% sodium hydroxide. Specifically, a gelatin solution comprising 1546.4 grams of gelatin, including 115.9 grams gelatin hydrolysate was developed. The solution was sterilized and a portion comprising 895.4 grams of the solution was dried, forming Sample A. The remaining portion of the gelatin solution (651 grams) was utilized to prepare Sample B. Specifically, to the remaining 651 grams of gelatin solution not incorporated into Sample A, 434 grams of calcium carbonate (Vicality™ Medium, mean particle size 1.9 microns, USP Grade) was added to attain a ratio of (dry) gelatin to (dry) calcium carbonate of approximately 3 to 2. The slurry was dried and the resulting combination was deemed Sample B. It is noted that the solutions were sterilized using a standard steam injection process whereby live, filtered steam is injected into the product, and the solution is maintained at a temperature of about 240° C. for a period of about 8 seconds, thereby killing any microorganisms present in the solution.

Subsequent to development, Sample A and Sample B were tested to determine the physical characteristics of each compound. Specifically, bloom strength, viscosity, moisture content, ash content, and calcium concentration were tested. The results are illustrated in Table 2 below.

TABLE 3
Physical Characteristics of Samples A and B.

| | | Viscosity | Moisture | | Calcium |
|---|---|---|---|---|---|
| | Bloom | (mp) | (%) | Ash (%) | (ppm) |
| Sample A | 125 | 24.8 | 12.8 | 0.81 | 48.2 |
| Sample B | 63 | 12.4 | 8.4 | 21.28 | 159740 |

After testing of Sample A and Sample B was performed to determine the physical characteristics of both gelatin solutions, each sample was incorporated into a gel mass solution for the development of gelatin coatings. Specifically, Sample A and Sample B were mixed with glycerin and water in the proportions found in Table 3, and subsequently de-bubbled to remove any air particles from the samples.

TABLE 4
Gel Mass Solutions

| | Weight of sample (g) | Glycerin (g) | Water (g) |
|---|---|---|---|
| Sample A | 895.4 | 412.5 | 754.6 |
| Sample B | 1085 | 300 | 548.8 |

The rapid-release agents (sodium carbonate and calcium carbonate) components were added to the preparations by means of the formation of striped regions on the gelatin layer comprising the rapid-release agent. A gel mass solution of Sample A gelatine, as described above was further adjusted to pH 8.9 by the addition of 2 Normal sodium hydroxide, added in portions. After each addition, a small aliquot of the gel mass solution was withdrawn and diluted to a gelatine concentration of 6.67 weight % and the pH of the aliquot was measured. This process was repeated until the measured pH was approximately 8.9. The resulting pH adjusted, undiluted gel mass was used to prepare gel mass mixtures using sodium carbonate and the gel mass solution of Sample B as follows:

TABLE 5
Gelatin Compositions

| Component % | Gelatine #1 | Gelatine #2 | Gelatine #3 | Gelatine #4 |
|---|---|---|---|---|
| Gelatine | 32.7 | 32.8 | 39.1 | 35.1 |
| CaCO3 | 6.4 | 11.1 | 0.0 | 7.1 |
| Na2CO3 | 6.2 | 4.6 | 5.0 | 4.8 |
| Glycerine | 16.4 | 16.4 | 19.6 | 17.9 |
| Water | 38.3 | 38.3 | 36.3 | 35.6 |

It is noted that some of the samples include both a water-insoluble (CaCO₃) and water-soluble (Na₂CO₃) rapid release agents in various ratios, while certain samples (Gelatine #3) include only the water-soluble rapid-release agent. As such, the experiment was designed to determine the effect of increasing the amount of water-soluble rapid-release agent compared to the water-insoluble rapid-release agent. Each of the samples above was coated on a polycarbonate plate using a coating gap of 700μ. The coated plates were dried in a constant humidity chamber at 22° C. and 50% R.H.

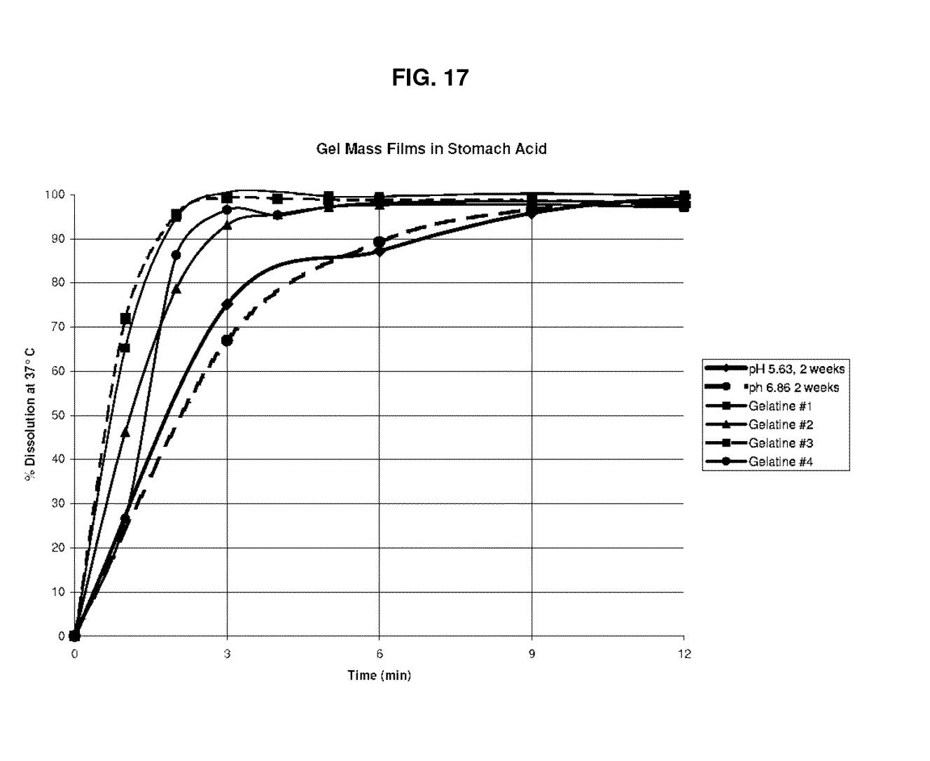

Subsequently, after gelatin sample preparation, the four gelatine samples, as well as two control samples were tested for solubility. Specifically, after drying for 2 weeks, a sample of the striped coating (usually approximately 1 cm×1 cm) was cut from the coating and the sample was tested for dissolution and disintegration by immersing the sample in a dissolution medium at 37° C. The dissolution medium was prepared to simulate stomach acid (without enzymes) according to the following procedure. Two grams of sodium chloride and 7.0 ml of concentrated HCl were dissolved in sufficient water to make 1000 ml. The pH of the dissolution medium was approximately 1.2. Gelatine Samples 1, 2, 3, and 4 disintegrated in less than 30 seconds, while the control samples disintegrated considerably slower. The dissolution profile for each sample is provided in FIG. 17 (FIG. 17).

FIG. 17 is a graph illustrating the dissolution time for the four different gelatin compositions including: a gelatin encapsulation composition containing calcium carbonate and sodium carbonate in a mass ratio of approximately 1:1 (6.4% to 6.2%, respectively); a gelatin encapsulation composition containing calcium carbonate and sodium carbonate in a mass ratio of approximately 2.4:1 (11.1% to 4.6%, respectively); a gelatin encapsulation composition containing only sodium carbonate (5.0% concentration); and a gelatin encapsulation composition containing calcium carbonate and sodium carbonate in a mass ratio of approximately 1.5:1 (7.1% to 4.8%, respectively). All four preparations dissolved significantly faster than the control films.

As illustrated in the graph, the greater the relative amount of water-soluble carbonate compared to the water-insoluble carbonate, the faster the dissolution profile. For instance, Gelatine #3, containing only water-soluble NA₂CO₃, without any CaCO₃ present, resulted in the fastest dissolution profile, followed by Gelatine #1 containing a calcium carbonate:sodium carbonate ratio of 1:1; Gelatine #4 containing a calcium carbonate:sodium carbonate ratio of 1.5:1, and Gelatine #2 containing a calcium carbonate:sodium carbonate ratio of 2.4:1, respectively. Accordingly, the results in FIG. 17 suggest that the greater the ratio of sodium carbonate (water-soluble release agent) to calcium carbonate (water-insoluble release agent), the faster the dissolution profile.

### Example 9

### Dissolution Testing of Soft Gel Capsules with Rapid-Release Agents

To further assess the dissolution properties of the various gelatin formulations discussed herein after exposure to temperature and humidity conditions, the following soft capsule experiment was conducted. Soft capsule gelatin compositions were prepared having viscosities at 60° C. between about 4,000 and about 10,000 mPas. The first gelatin composition included the bone gelatin without any further modifications at 44.00 weight percent, polysorb at 11.00%, glycerol (85%) at 11.00%, and water at 34.00% (“Std Bone Gelatine”). The second gelatin composition included bone gelatin at 37.67%, hydrolyzed bone gelatin having a molecular weight of about 500 Daltons at 4.96%, calcium carbonate (CaCO3) at 6.38%, polysorb at 10.02%, glycerol (85%) at 10.02%, and water at 30.97% (“CaCO3”). The third gelatin composition included bone gelatin at 37.11%, hydrolyzed bone gelatin having a molecular weight of about 500 Daltons at 4.88%, calcium carbonate (CaCO3) at 6.28%, Potassium bicarbonate at 1.47%, polysorb at 9.87%, glycerol (85%) at 9.87%, and water at 30.51% (“Band”). The fourth gelatin composition included bone gelatine at 40.23%, hydrolyzed bone gelatin having a molecular weight of about 500 Daltons at 5.08%, 1.0 Normal Sodium hydroxide solution at 7.40%, polysorb at 9.87%, glycerol (85%) at 9.87%, and water at 23.11% (“HipH”).

The four gelatin compositions were utilized to make soft capsules on a Modified Chan Sung soft capsule machine Type M3 having dual cavity dye rolls to make 7.5 oval capsules and operating at 2.5 rpm with 30 minute tumble drying followed by one week room drying at about 25° C. and about 35% RH. The configuration of the shells of some of the capsules was modified to create a narrow region of the Band formulation in capsules that had most of the shell composition incorporating the HipH formulation, utilizing a precision metering pump to deliver the Band formulation into the nip of the spreader box of the encapsulation machine using a needle. The flow rate of the Band formulation was adjusted to create different widths of the region. A flow rate of 0.16 ml/min produced a narrow region (“Narrow Band”) and a flow rate of 0.40 ml/min produced a broader region (“Medium Band”). In addition to the banded capsules, formulations were prepared having only one type of gelatin composition, i.e., all Std Bone Gelatine, all CaCO3, and all HipH, for a total of five capsule configurations.

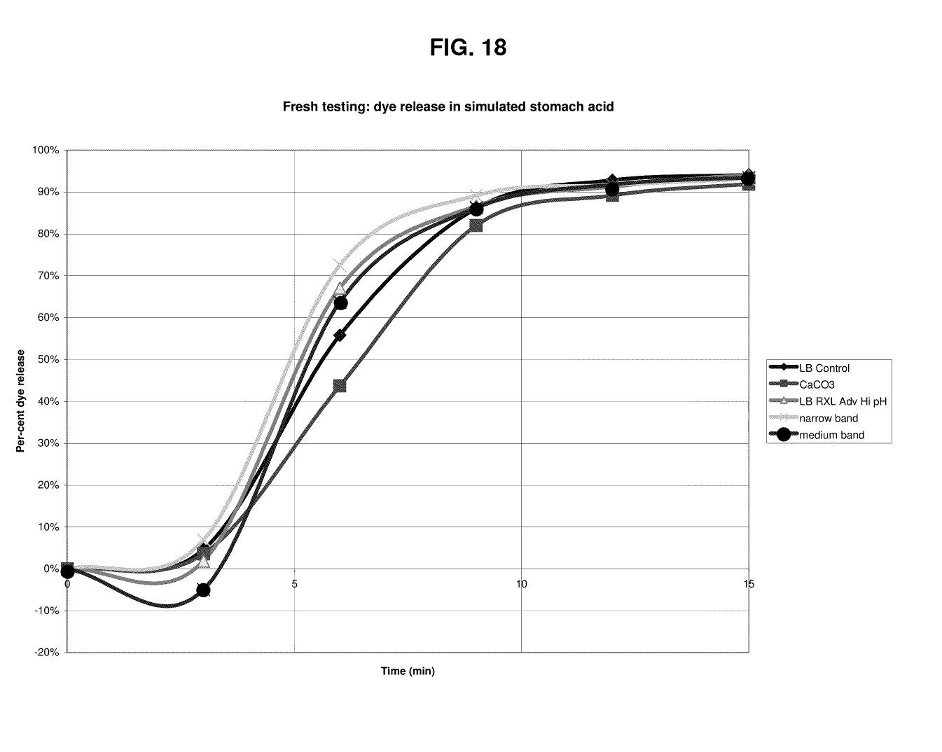

All capsules were filled with a liquid formulation comprising polyethylene-glycol at 96.51%, glycerol at 2.99%, and brilliant blue dye at 0.50% (the fill). The five capsule shell configurations were tested to determine the dissolution profile over time in an acidic dissolution medium. In each case, the dissolution media were monitored spectrophotometrically to observe the appearance of brilliant blue dye in the media. The test assessed the dissolution profile for fresh capsules of the five shell configurations in simulated gastric fluid (at a pH of approximately 1.3, and in the absence of any enzymes). The results of testing of fresh capsules after drying are illustrated in FIG. 18.

The results illustrated in FIG. 18 show that the “CaCO3” formulation released dye slower than normal gelatine (“Std Bone Gelatine”). The narrow and normal band formulations released dye faster than the Std Bone Gelatine formulation, and the fastest release was observed for the narrow band configuration.

Having described the invention in detail, it will be apparent that modifications and variations are possible. Those of skill in the art should, in light of the present disclosure, appreciate that many changes could be made in the specific embodiments that are disclosed and still obtain a like or similar result without departing from the spirit and scope of the invention, therefore all matter set forth is to be interpreted as illustrative and not in a limiting sense.

What is claimed is:

1. A gelatin capsule composition comprising an outer surface coating and a core, the outer surface coating comprising:
a. one or more first distinct regions comprising a gelatin component; and,
b. one or more second distinct regions comprising a rapid-release agent.

2. The composition of claim 1, wherein the one or more first distinct regions comprising the gelatin component and the one or more second distinct regions comprising the rapid-release agent further comprise a gelatin hydrolysate.

3. The composition of claim 2, wherein the gelatin hydrolysate has an average molecular weight ranging from about 100 to about 2000 Daltons.

4. The composition of claim 1, wherein the gelatin component has an average molecular weight ranging from about 50,000 Daltons to about 300,000 Daltons.

5. The composition of claim 1, wherein the one or more second distinct regions comprising a rapid-release agent span the depth of the outer surface coating from the proximal surface to the distal surface.

6. The composition of claim 1, wherein the one or more second distinct regions comprising a rapid-release agent are dispersed across the gelatin capsule composition in a uniform pattern whereby the length and width of each second distinct region does not vary more than 10% relative to one another.

7. The composition of claim 1, wherein the one or more second distinct regions comprising a rapid-release agent are dispersed across the gelatin capsule composition in a non-uniform pattern whereby the length and width of each second distinct region varies more than 10% relative to one another.

8. The composition of claim 1, wherein the rapid-release agent comprises a water-insoluble carbonate salt, a water-soluble carbonate salt, and combinations thereof.

9. The composition of claim 8, wherein the water-insoluble carbonate salt comprises bismuth subcarbonate, calcium carbonate, cobalt carbonate, lanthanum carbonate, lead carbonate, lithium carbonate, magnesium carbonate, manganese carbonate, nickel (II) carbonate, silver carbonate, strontium carbonate, and combinations thereof.

10. The composition of claim 9, wherein the water-insoluble rapid-release agent consists of calcium carbonate.

11. The composition of claim 8, wherein the water-soluble carbonate salt comprises sodium carbonate, potassium carbonate, ammonium carbonate, sodium bicarbonate, potassium bicarbonate, ammonium bicarbonate, lithium carbonate, and combinations thereof.

12. The composition of claim 8, wherein the water-insoluble carbonate salt is essentially insoluble at a pH ranging from about 6 to about 8, and wherein the rapid-release agent dissociates at a pH ranging from 0 to about 3.

13. The composition of claim 8, wherein the water-soluble carbonate salt is at least partially soluble in water, and wherein the rapid-release agent dissociates at a pH ranging from 0 to about 3.

14. The composition of claim 1, wherein the gelatin capsule composition degrades essentially completely in less than 15 minutes at a pH ranging between 0 and about 3.

15. The composition of claim 1, wherein the outer surface coating further comprises a plasticizer.

16. The composition of claim 15, wherein the plasticizer comprises dibutyl sebacate, diethyl phthalate, glycerine, polyethylene glycol, propylene glycol, sorbitol, sorbitans, erythritol, triacetin, triethyl citrate, water, and combinations thereof.

17. The composition of claim 1, wherein the gelatin component of the one or more first distinct regions comprises a combination of a gelatin, a plasticizer, and water.

18. The composition of claim 17, wherein the gelatin component of the one or more first distinct regions comprises about 25% to about 55% by weight of the one or more first distinct regions of the gelatin, about 10% to about 30% by weight of the one or more first distinct regions of the plasticizer, and about 15% to about 45% by weight of the one or more first distinct regions of water.

19. The composition of claim 17, wherein the gelatin component of the one or more first distinct regions comprises about 35% to about 45% by weight of the one or more first distinct regions of the gelatin, about 16% to about 24% by weight of the one or more first distinct regions of the plasticizer, and about 20% to about 30% by weight of the one or more first distinct regions of the water.

20. The composition of claim 1, wherein the gelatin component of the one or more first distinct regions comprises a combination of a gelatin and water.

21. The composition of claim 20, wherein the gelatin component comprises about 5% to about 30% gelatin and about 70% to about 95% water.

22. The composition of claim 20, wherein the gelatin component comprises about 10% to about 20% gelatin and about 80% to about 90% water.

23. The composition of claim 2, wherein the outer surface coating comprises a mass ratio of the gelatin component to the gelatin hydrolysate ranging from about 3:1 to about 99:1.

24. The composition of claim 2, wherein the outer surface coating comprises a mass ratio of the gelatin component to the gelatin hydrolysate ranging from about 4:1 to about 19:1.

25. The composition of claim 1, wherein the one or more second distinct regions comprising a rapid-release agent further comprise a gelatin, a plasticizer, and water.

26. The composition of claim 25, wherein the one or more second distinct regions comprise about 20% to about 50% by weight of the one or more second distinct regions of the gelatin, about 1% to about 25% by weight of the one or more second distinct regions of the rapid-release agent, about 10% to about 30% by weight of the one or more second distinct regions of the plasticizer, and about 20% to about 40% by weight of the one or more second distinct regions of water.

27. The composition of claim 25, wherein the one or more second distinct regions comprise about 30% to about 45% by weight of the one or more second distinct regions of the gelatin, about 4% to about 16% by weight of the one or more second distinct regions of the rapid-release agent, about 16% to about 24% by weight of the one or more second distinct regions of the plasticizer, and about 26% to about 34% by weight of the one or more second distinct regions of water.

28. The composition of claim 25, wherein the one or more second distinct regions comprise a mass ratio of the rapid-release agent to the gelatin ranging from about 1:1 to about 1:20.

29. The composition of claim 25, wherein the one or more second distinct regions comprise a mass ratio of the rapid-release agent to the gelatin ranging from about 1:2 to about 1:9.

30. The composition of claim 1, wherein the core comprises a solid formulation and a liquid formulation.

31. The composition of claim 1, wherein the core comprises an active pharmaceutical ingredient, and, optionally, one or more pharmaceutically acceptable excipients.

32. The composition of claim 1, wherein the one or more first distinct regions further comprise a rapid-release agent, and
wherein the rapid-release agent of the one or more first distinct regions is present in a concentration less than the concentration of the rapid-release agent of the one or more second distinct regions.

33. The composition of claim 1, wherein the gelatin component comprises Type A gelatin, Type B gelatin, and combinations thereof.

34. The composition of claim 1, wherein the one or more second distinct regions comprising a rapid-release agent have the appearance of stripes, bars, bands, streaks, strips, rows, columns, spots, flecks, striations, belts, ribbons, veins, dashes, ridges, strains, and combinations thereof

35. A gelatin capsule composition comprising an outer surface coating and a core, the outer surface coating comprising:
a. one or more first distinct regions comprising a gelatin component and a gelatin hydrolysate; and,
b. one or more second distinct regions comprising a rapid-release agent, a gelatin component, and a gelatin hydrolysate,
wherein the one or more second distinct regions span the depth of the outer surface coating from the proximal surface to the distal surface.

36. The composition of claim 35, wherein the gelatin hydrolysate has an average molecular weight ranging from about 100 to about 2000 Daltons.

37. The composition of claim 35, wherein the gelatin component of the one or more first distinct regions and the one or more second distinct regions has an average molecular weight ranging from about 50,000 Daltons to about 300,000 Daltons.

38. The composition of claim 35, wherein the rapid-release agent comprises a water-insoluble carbonate salt, a water-soluble carbonate salt, and combinations thereof.

39. The composition of claim 38, wherein the water-insoluble carbonate salt comprises bismuth subcarbonate, calcium carbonate, cobalt carbonate, lanthanum carbonate, lead carbonate, lithium carbonate, magnesium carbonate, manganese carbonate, nickel (II) carbonate, silver carbonate, strontium carbonate, and combinations thereof.

40. The composition of claim 39, wherein the water-insoluble rapid-release agent consists of calcium carbonate.

41. The composition of claim 38, wherein the water-soluble carbonate salt comprises sodium carbonate, potassium carbonate, ammonium carbonate, sodium bicarbonate, potassium bicarbonate, ammonium bicarbonate, lithium carbonate, and combinations thereof.

42. The composition of claim 38, wherein the water-insoluble carbonate salt is essentially insoluble at a pH ranging from about 6 to about 8, and wherein the rapid-release agent dissociates at a pH ranging from 0 to about 3.

43. The composition of claim 38, wherein the water-soluble carbonate salt is at least partially soluble in water, and wherein the rapid-release agent dissociates at a pH ranging from 0 to about 3.

44. The composition of claim 35, wherein the gelatin component of the one or more first distinct regions and the one or more second distinct regions comprise a combination of a gelatin, a plasticizer, and water.

45. The composition of claim 44, wherein the one or more first distinct regions comprise about 25% to about 55% by weight of the one or more first distinct regions of the gelatin, about 0.01% to about 20% by weight of the one or more first distinct regions of the gelatin hydrolysate, about 10% to about 30% by weight of the one or more first distinct regions of the plasticizer, and about 15% to about 45% by weight of the one or more first distinct regions of water.

46. The composition of claim 44, wherein the one or more first distinct regions comprise about 35% to about 48% by weight of the one or more first distinct regions of the gelatin, about 0.1% to about 10% by weight of the one or more first distinct regions of the gelatin hydrolysate, about 16% to about 24% by weight of the one or more first distinct regions of the plasticizer, and about 20% to about 30% by weight of the one or more first distinct regions of water.

47. The composition of claim 44, wherein the one or more second distinct regions comprise about 20% to about 50% by weight of the one or more second distinct regions of the gelatin, about 0.1% to about 20% by weight of the one or more second distinct regions of the gelatin hydrolysate, about 1% to about 25% by weight of the one or more second distinct regions of the rapid-release agent, about 10% to about 30% by weight of the one or more second distinct regions of the plasticizer, and about 20% to about 40% by weight of the one or more second distinct regions of water.

48. The composition of claim 44, wherein the one or more second distinct regions comprise about 30% to about 45% by weight of the one or more second distinct regions of the gelatin, about 1% to about 10% by weight of the one or more second distinct regions of the gelatin hydrolysate, about 4% to about 16% by weight of the one or more second distinct regions of the rapid-release agent, about 16% to about 24% by weight of the one or more second distinct regions of the plasticizer, and about 26% to about 34% by weight of the one or more second distinct regions of water.

49. The composition of claim 35, further comprising an active pharmaceutical ingredient.

50. A pharmaceutical gelatin capsule composition comprising an outer surface coating and a core, the outer surface coating comprising:
a. one or more first distinct regions comprising about 35% to about 45% by weight of the one or more first distinct regions of a gelatin, about 3% to about 7% by weight of the one or more first distinct regions of a gelatin hydrolysate having an average molecular weight ranging from about 100 to about 2000 Daltons, about 18% to about 22% by weight of the one or more first distinct regions of a plasticizer, and about 20% to about 26% by weight of the one or more first distinct regions of water; and,
b. one or more second distinct regions comprising about 34% to about 40% by weight of the one or more second distinct regions of a gelatin, about 3% to about 7% by weight of the one or more second distinct regions of a gelatin hydrolysate having an average molecular weight ranging from about 100 to about 2000 Daltons, about 5% to about 10% by weight of the one or more second distinct regions of a rapid release agent, about 18% to about 22% by weight of the one or more second distinct regions of a plasticizer, and about 28% to about 32% by weight of the one or more second distinct regions of water,
wherein the core comprises an active pharmaceutical ingredient,
wherein the one or more second distinct regions span the depth of the outer surface coating from the proximal surface to the distal surface, and
wherein the rapid release agent comprises bismuth subcarbonate, calcium carbonate, cobalt carbonate, lanthanum carbonate, lead carbonate, magnesium carbonate, manganese carbonate, nickel (II) carbonate, silver carbonate, strontium carbonate, sodium carbonate, potassium carbonate, ammonium carbonate, sodium bicarbonate, potassium bicarbonate, ammonium bicarbonate, lithium carbonate, and combinations thereof.

51. The composition of claim 50, wherein the one or more first distinct regions further comprises a rapid release agent, and
wherein the concentration of the rapid-release agent included in the one or more first distinct regions is less than the concentration of the rapid release agent included in the one or more second distinct regions.

52. A gelatin capsule composition comprising an outer surface coating, a subcoating, and a core, the outer surface coating comprising:
a. one or more first distinct regions comprising a gelatin component; and,
b. one or more second distinct regions comprising a rapid-release agent,
wherein the subcoating is applied to the distal surface of the core and surrounds a substantial portion of the core, and
wherein the outer surface coating is applied to the distal surface of the subcoating.

53. The composition of claim 52, wherein the subcoating comprises a gelatin component, a gelatin hydrolysate, a polymeric material, and combinations thereof.

54. The composition of claim 52, wherein the subcoating includes one or more passageways for the release of the contents of the core.

55. The composition of claim 52, wherein the subcoating comprises two or more layers having distinct release profiles.

56. The composition of claim 52, wherein the core comprises an active pharmaceutical ingredient.

57. A gelatin capsule composition comprising an enteric coating, a core surface coating, and a core, the core surface coating comprising:
a. one or more first distinct regions comprising a gelatin component; and,
b. one or more second distinct regions comprising a rapid-release agent,
wherein the enteric coating is applied to the distal surface of the core surface coating,
wherein the core surface coating is applied to the distal surface of the core, and
wherein the enteric coating is not soluble at pH levels ranging from 0 to about 3 and dissolves at pH levels of greater than or equal to about 5.0.

58. The composition of claim 57, wherein the enteric coating comprises a gelatin component, a gelatin hydrolysate, a polymeric material, and combinations thereof.

59. The composition of claim 57, wherein the core comprises an active pharmaceutical ingredient.

60. A method for manufacturing a gelatin capsule composition comprising an outer surface coating and a core containing an active pharmaceutical ingredient, the outer surface coating comprising one or more first distinct regions comprising a gelatin component, and one or more second distinct regions comprising a rapid-release agent, the method comprising:
a. dissolving a gelatin component in an aqueous medium to create an aqueous gelatin solution;
b. incorporating a second solution comprising a rapid-release agent into the aqueous gelatin solution to produce one or more first distinct regions comprising the aqueous gelatin solution and one or more second distinct regions comprising the second solution comprising the rapid-release agent; and,
c. encapsulating the core containing an active pharmaceutical ingredient while maintaining the one or more first distinct regions and the one or more second distinct regions.

61. The method of claim 60, wherein the encapsulating step comprises an extrusion process.

62. The method of claim 60, wherein the gelatin component of step (a) comprises a combination of a gelatin having a molecular weight ranging from about 50,000 Daltons to about 300,000 Daltons and a gelatin hydrolysate having a molecular weight ranging from about 100 Daltons to about 2000 Daltons.

63. The method of claim 60, wherein the one or more second distinct regions further comprises a gelatin component and the mass ratio of the rapid-release agent to the gelatin component ranges from about 1:1 to about 1:20.

64. The method of claim 60, wherein step (a) further comprises the addition of a plasticizer.

65. The method of claim 64, wherein the plasticizer comprises dibutyl sebacate, diethyl phthalate, glycerine, polyethylene glycol, propylene glycol, sorbitol, erythritol, triacetin, triethyl citrate, water, and mixtures thereof.

66. The method of claim 60, wherein the gelatin component comprises a combination of a gelatin and a plasticizer.

67. The method of claim 62, wherein the mass ratio of the gelatin having a molecular weight ranging from about 50,000 Daltons to about 300,000 Daltons to the gelatin hydrolysate having a molecular weight ranging from about 100 Daltons to about 2000 Daltons ranges from about 3:1 to about 99:1.

68. The method of claim 60, wherein the gelatin component of step (a) comprises the combination of a gelatin, a plasticizer, and an aqueous medium, and
wherein step (a) comprises mixing about 30% to about 55% by weight of the one or more first distinct regions of the gelatin, about 10% to about 30% by weight of the one or more first distinct regions of the plasticizer, and about 15% to about 45% by weight of the one or more first distinct regions of the aqueous medium.

69. The method of claim 68, wherein step (a) comprises mixing about 35% to about 45% by weight of the one or more first distinct regions of the gelatin, about 0.1% to about 20% by weight of the one or more first distinct regions of a gelatin hydrolysate having an average molecular weight ranging from about 100 to about 2000 Daltons, about 10% to about 30% by weight of the one or more first distinct regions of a plasticizer, and about 25% to about 45% by weight of the one or more first distinct regions of the aqueous medium.

70. The method of claim 60, wherein the second solution of step (b) comprises a rapid-release agent, a gelatin component, a plasticizer, and an aqueous medium.

71. The method of claim 70, wherein the gelatin component of step (b) comprises a combination of a gelatin having a molecular weight ranging from about 50,000 Daltons to about 300,000 Daltons and a gelatin hydrolysate having a molecular weight ranging from about 100 Daltons to about 2000 Daltons.

72. The method of claim 71, wherein the second solution of step (b) comprises about 20% to about 50% by weight of the second solution of the gelatin, about 0.01% to about 20% by weight of the second solution of the gelatin hydrolysate, about 1% to about 25% by weight of the second solution of the rapid-release agent, about 10% to about 30% by weight of the second solution of the plasticizer, and about 20% to about 40% by weight of the second solution of water.

73. The method of claim 60, wherein the aqueous medium comprises water.

74. The method of claim 60, wherein the one or more first distinct regions comprising an aqueous gelatin solution and the one or more second distinct regions comprising a rapid-release agent are produced by feeding the aqueous gelatin solution and the second solution comprising the rapid-release agent through a static mixer.

75. A gelatin composition comprising an outer surface coating and a core, wherein the outer surface coating comprises a rapid-release agent and a gelatin component.

76. The composition of claim 75, wherein the composition further comprises a gelatin hydrolysate having a molecular weight ranging from about 100 Daltons to about 2000 Daltons.

77. The composition of claim 75, wherein the rapid-release agent comprises water-soluble carbonate salts, water-insoluble carbonate salts, and combinations thereof.

78. The composition of claim 77, wherein the water-soluble rapid release agent comprises sodium carbonate, potassium carbonate, ammonium carbonate, sodium bicarbonate, potassium bicarbonate, ammonium bicarbonate, lithium carbonate, and combinations thereof.

79. The composition of claim 77, wherein the water-soluble carbonate salt is at least partially soluble in water, and wherein the rapid-release agent dissociates at a pH ranging from 0 to about 3.

80. The composition of claim 77, wherein the water-insoluble carbonate salt comprises bismuth subcarbonate, calcium carbonate, cobalt carbonate, lanthanum carbonate, lead carbonate, lithium carbonate, magnesium carbonate, manganese carbonate, nickel (II) carbonate, silver carbonate, strontium carbonate, and combinations thereof.

81. The composition of claim 80, wherein the water-insoluble rapid-release agent consists of calcium carbonate.

82. The composition of claim 75, wherein the composition further comprises a mass ratio of the rapid-release agent to the gelatin component ranging from about 1:1 to about 1:20.

83. The composition of claim 82, wherein the mass ratio of the rapid-release agent to the gelatin component ranges from about 1:2 to about 1:15.

84. The composition of claim 82, wherein the mass ratio of the rapid-release agent to the gelatin component ranges from about 1:4 to about 1:9.

85. The composition of claim 75, wherein the contents of the core are released in less than 15 minutes at a pH ranging between 0 and about 3.

86. The composition of claim 75, further comprising a plasticizer.

87. The composition of claim 86, wherein the plasticizer comprises dibutyl sebacate, diethyl phthalate, glycerine, polyethylene glycol, propylene glycol, sorbitol, erythritol, triacetin, triethyl citrate, water, and mixtures thereof.

88. The composition of claim 75, wherein the gelatin component comprises a combination of a gelatin, a plasticizer, and water.

89. The composition of claim 88, wherein the gelatin component comprises about 25% to about 55% by weight of the gelatin component of the gelatin, about 15% to about 30% by weight of the gelatin component of the plasticizer, and about 25% to about 40% by weight of the gelatin component of the water.

90. The composition of claim 88, wherein the gelatin component comprises about 35% to about 45% by weight of the gelatin component of the gelatin, about 20% to about 25% by weight of the gelatin component of the plasticizer, and about 30% to about 35% by weight of the gelatin component of the water.

91. The composition of claim 75, wherein the gelatin component comprises a combination of a gelatin and water.

92. The composition of claim 91, wherein the gelatin component comprises about 5% to about 30% gelatin and about 70% to about 95% water.

93. The composition of claim 91, wherein the gelatin component comprises about 10% to about 20% gelatin and about 80% to about 90% water.

94. The composition of claim 76, wherein the composition further comprises a mass ratio of the gelatin component to the gelatin hydrolysate ranging from about 3:1 to about 99:1.

95. The composition of claim 76, wherein the composition further comprises a mass ratio of the gelatin component to the gelatin hydrolysate ranging from about 4:1 to about 19:1.

96. The composition of claim 76, wherein the composition further comprises a mass ratio of the gelatin component to the gelatin hydrolysate ranging from about 5:1 to about 13:1.

97. A gelatin composition comprising an outer surface coating and a core, the outer surface coating comprising:
a. a rapid-release agent;
b. a gelatin component; and
c. a gelatin hydrolysate,
wherein the composition comprises a mass ratio of the rapid-release agent to the gelatin component ranging from about 1:1 to about 1:20,
wherein the gelatin hydrolysate has a molecular weight ranging from about 100 Daltons to about 2000 Daltons, and
wherein the composition comprises a mass ratio of the gelatin component to the gelatin hydrolysate ranging from about 3:1 to about 99:1.

98. A gelatin composition comprising a rapid-release agent and a gelatin component, prepared by the process comprising:
a. dissolving the gelatin component in an aqueous medium to form an aqueous gelatin solution;
b. mixing the rapid-release agent with the aqueous gelatin solution prior to capsule formation; and
c. incorporating the combination of the aqueous gelatin solution and rapid-release agent into a gelatin capsule forming machine.

99. The gelatin composition of claim 98, wherein step (b) comprises mixing the rapid-release agent with the aqueous gelatin solution prior to capsule formation comprises an in-line mixing process.

100. The gelatin composition of claim 99, wherein the in-line mixing process comprises the use of a static mixer.

101. The gelatin composition of claim 98, wherein the composition further comprises a gelatin hydrolysate having a molecular weight ranging from about 100 Daltons to about 2000 Daltons.

102. The gelatin composition of claim 98, wherein the water-soluble rapid-release agent comprises bismuth subcarbonate, calcium carbonate, cobalt carbonate, lanthanum carbonate, lead carbonate, lithium carbonate, magnesium carbonate, manganese carbonate, nickel (II) carbonate, silver carbonate, strontium carbonate, lithium carbonate, sodium carbonate, potassium carbonate, ammonium carbonate, sodium bicarbonate, potassium bicarbonate, ammonium bicarbonate, lithium carbonate, and combinations thereof.

103. A method for manufacturing a gelatin composition comprising a rapid release agent, comprising the steps of:
a. dissolving a gelatin component in an aqueous medium to form an aqueous gelatin solution;
b. mixing a rapid-release agent with the aqueous gelatin solution prior to capsule formation; and
c. incorporating the combination of the aqueous gelatin solution and rapid-release agent into a gelatin capsule forming machine.

104. The method of claim 103, wherein step (b) comprising mixing the water-soluble rapid-release agent with the aqueous gelatin solution prior to capsule formation comprises an in-line mixing process.

105. The method of claim 103, wherein the gelatin component of step (a) comprises a combination of a gelatin having a molecular weight ranging from about 50,000 Daltons to about 300,000 Daltons and a gelatin hydrolysate having a molecular weight ranging from about 100 Daltons to about 2000 Daltons.

106. The method of claim 103, wherein the rapid-release agent comprises bismuth subcarbonate, calcium carbonate, cobalt carbonate, lanthanum carbonate, lead carbonate, lithium carbonate, magnesium carbonate, manganese carbonate, nickel (II) carbonate, silver carbonate, strontium carbonate, lithium carbonate, sodium carbonate, potassium carbonate, ammonium carbonate, sodium bicarbonate, potassium bicarbonate, ammonium bicarbonate, lithium carbonate, and combinations thereof.

107. The method of claim 103, wherein the water-soluble rapid-release agent is at least partially soluble in water, and wherein the rapid-release agent dissociates at a pH ranging from 0 to about 3.

108. The method of claim 103, wherein the mass ratio of the rapid-release agent to the gelatin component ranges from about 1:1 to about 1:20.

109. The method of claim 103, wherein the gelatin composition degrades essentially completely in less than 15 minutes at a pH ranging between 0 and about 3.

110. The method of claim 105, wherein the mass ratio of the gelatin having a molecular weight ranging from about 50,000 Daltons to about 300,000 Daltons to the gelatin hydrolysate having a molecular weight ranging from about 100 Daltons to about 2000 Daltons ranges from about 3:1 to about 99:1.

111. The method of claim 105, wherein the mass ratio of the gelatin having a molecular weight ranging from about 50,000 Daltons to about 300,000 Daltons to the gelatin hydrolysate having a molecular weight ranging from about 100 Daltons to about 2000 Daltons ranges from about 4:1 to about 19:1.

112. The method of claim 105, wherein the mass ratio of the gelatin having a molecular weight ranging from about 50,000 Daltons to about 300,000 Daltons to the gelatin hydrolysate having a molecular weight ranging from about 100 Daltons to about 2000 Daltons ranges from about 5:1 to about 13:1.

113. The method of claim 103, wherein step (a) comprises dissolving about 0.01% to about 30% of the gelatin component by weight of the aqueous gelatin solution in about 40% to about 99.9% of the aqueous medium by weight of the aqueous gelatin solution.

114. The method of claim 103, wherein step (a) comprises dissolving about 10% to about 20% of the gelatin component by weight of the combined aqueous gelatin solution in about 70% to about 90% of the aqueous medium by weight of the combined aqueous gelatin solution.

115. The method of claim 105, wherein step (a) comprises dissolving about 10% to about 20% by weight of the gelatin having a molecular weight ranging from about 50,000 Daltons to about 300,000 Daltons and about 1% to about 5% by weight of the gelatin hydrolysate having a molecular weight ranging from about 100 Daltons to about 2000 Daltons in about 70% to about 90% aqueous medium by weight of the combined aqueous gelatin solution.

116. The method of claim 103, wherein the aqueous medium comprises water.
